SEQUENCE LISTING (9 sequences)

SEQ ID NO: 1 - DNA, 20 bp, synthetic construct
  source 1..20: mol_type = other DNA
ctcacccagcacgaaggaaa

SEQ ID NO: 2 - DNA, 20 bp, synthetic construct
  source 1..20: mol_type = other DNA
ccatggtggcgcatccgtac

SEQ ID NO: 3 - DNA, 20 bp, synthetic construct
  source 1..20: mol_type = other DNA
gctggcctgcgcggtcccac

SEQ ID NO: 4 - DNA, 20 bp, synthetic construct
  source 1..20: mol_type = other DNA
tccttggtgacccacttaca

SEQ ID NO: 5 - DNA, 20 bp, synthetic construct
  source 1..20: mol_type = other DNA
tctggagcaggatagggtcc

SEQ ID NO: 6 - DNA, 20 bp, synthetic construct
  source 1..20: mol_type = other DNA
tgtatacacaggtgaggact

SEQ ID NO: 7 - DNA, 20 bp, synthetic construct
  source 1..20: mol_type = other DNA
gcaggctctggagcaggata

SEQ ID NO: 8 - DNA, 120 bp, synthetic construct
  source 1..120: mol_type = other DNA
gccctgggtgtggtttgccaacctgtcaatgaaggcgtggcttggaggtcgaattcgggaccctaccctgctccagagcctgctgaacgtgagcccactgtacagacagggctgccgcag

SEQ ID NO: 9 - DNA, 120 bp, synthetic construct
  source 1..120: mol_type = other DNA
tcagtgtggacacctgtatgcctacgactggattagtatcccactggtgtgtacacaggtgaggactagtctggtgaggctgcccttttgggaaactgaggctagaaggaccaaggaagc

(19) United States
(12) Patent Application Publication
(10) Pub. No.: US 2024/0043848 A1
(43) Pub. Date: Feb. 8, 2024

(54) RESCUE STRATEGIES FOR BEST1 LOSS- AND GAIN-OF-FUNCTION MUTATIONS

(71) Applicants: The Trustees of Columbia University in the City of New York, New York, NY (US); University of Rochester, Rochester, NY (US)

(72) Inventors: Tingting Yang, New York, NY (US); Stephen H. Tsang, New York, NY (US); Yu Zhang, New York, NY (US)

(21) Appl. No.: 18/485,930

(22) Filed: Oct. 12, 2023

Related U.S. Application Data
(63) Continuation of application No. PCT/US2022/024622, filed on Apr. 13, 2022.
(60) Provisional application No. 63/174,090, filed on Apr. 13, 2021.

(51) Int. Cl. C12N 15/113; C12N 15/85; A61K 48/00; C12N 9/22; C07K 14/705

(52) U.S. Cl. CPC C12N 15/1138; C12N 15/85; A61K 48/005; C12N 9/22; C07K 14/705; C12N 2310/20; C12N 2710/14043

(57) ABSTRACT

The present disclosure relates to methods, compositions, and systems for rescuing gene function and the treatment and prevention of a disease or disorder (e.g., bestrophinopathies).

### CROSS-REFERENCE TO RELATED APPLICATIONS

This application is a continuation of International Application No. PCT/US2022/024622, filed Apr. 13, 2022, which claims the benefit of U.S. Provisional Application No. 63/174,090, filed Apr. 13, 2021, the contents of which are herein incorporated by reference in its entirety.

### STATEMENT REGARDING FEDERALLY SPONSORED RESEARCH OR DEVELOPMENT

This invention was made with government support under EY028758 and GM127652 awarded by the National Institutes of Health. The government has certain rights in the invention.

### TECHNICAL FIELD

The present disclosure relates to methods, compositions, and systems for rescuing gene function and the treatment and prevention of a disease or disorder (e.g., bestrophinopathies).

### SEQUENCE LISTING STATEMENT

The text of the computer readable sequence listing filed herewith, titled “COLUM-40036.302.xml”, created Oct. 12, 2023, having a file size of 9,182 bytes, is hereby incorporated by reference in its entirety.

### BACKGROUND OF THE INVENTION

Genetic mutation of the human BEST1 gene, which encodes a Ca²⁺-activated Cl⁻ channel (BEST1) predominantly expressed in retinal pigmented epithelium (RPE), causes a spectrum of retinal degenerative disorders commonly known as bestrophinopathies. To date, the pathological mechanisms of BEST1 mutations remain unclear, thus, there are no effective treatment or prevention strategies.

### SUMMARY OF THE INVENTION

Provided herein are methods for rescuing gene function and the treatment and prevention of diseases and disorders (e.g., bestrophinopathies). In some embodiments, the methods comprise introducing into a cell an effective amount of: a Clustered Regularly Interspaced Short Palindromic Repeats (CRISPR)-Cas system, or one or more nucleic acids encoding the CRISPR-Cas system, configured to knockout or at least partially silence both alleles of a target endogenous gene; and a nucleic acid encoding an exogenous functional version of the target endogenous gene.

In some embodiments, the CRISPR-Cas system comprising at least one Cas protein and at least one gRNA, wherein each gRNA is configured to hybridize to a portion of the nucleic acid sequence encoding the target endogenous gene. In some embodiments, the at least one gRNA is configured to not hybridize to the exogenous functional version of the target endogenous gene. In some embodiments, the exogenous functional version of the target endogenous gene comprises a nucleic acid sequence different from that of the target endogenous gene and encodes for a polypeptide comprising an amino acid sequence at least 90% identical to that of the wild-type version of the target endogenous gene.

In some embodiments, the one or more nucleic acids comprises one or more messenger RNAs, one or more vectors, or a combination thereof.

In some embodiments, the CRISPR-Cas system is a CRISPR interference (CRISPRi) system. In some embodiments, the CRISPRi system comprises: at least one Cas protein; at least one gRNA, wherein each gRNA is configured to hybridize to a portion of the nucleic acid sequence encoding the target endogenous gene; and a transcriptional repressor. In some embodiments, the Cas protein and the transcriptional repressor are provided as a fusion protein, or a nucleic acid encoding thereof. In some embodiments, the transcriptional repressor and the Cas protein or the at least one gRNA each comprise one half of a binding pair from a recruitment system. In some embodiments, the Cas protein is catalytically dead. In some embodiments, the Cas protein is Cas9, Cas12a, and Cas14.

In some embodiments, the Cas protein, the at least one gRNA, and when included, the transcriptional repressor are provided on a single nucleic acid. In some embodiments, the single nucleic acid is a vector. In some embodiments, the single nucleic acid is a baculovirus or lentivirus vector.

In some embodiments, the target endogenous gene is a disease-associated gene. In some embodiments, at least one allele of the target endogenous gene has a gain of function mutation. In some embodiments, at least one allele of the target endogenous gene has a loss of function mutation. In some embodiments, the target endogenous gene is BEST1. In some embodiments, the BEST1 gene comprises a D203A, I205T or Y236C mutation.

In some embodiments, the cell is in vivo. In some embodiments, introducing into a cell comprises administering to a subject.

In some embodiments, the subject has or is suspected to have an ocular disease or disorder. In some embodiments, the subject has or is suspected to have a neurodegenerative disease. In some embodiments, the disease or disorder comprises Best vitelliform macular dystrophy (BVMD), autosomal recessive bestrophinopathy (ARB), adult-onset vitelliform dystrophy (AVMD), autosomal dominant vitreoretinochoroidopathy (ADVIRC), or retinitis pigmentosa (RP). In some embodiments, the method treats or prevents the disease or disorder in the subject.

Also provided herein are systems comprising: a Clustered Regularly Interspaced Short Palindromic Repeats (CRISPR)-Cas system, or one or more nucleic acids encoding the CRISPR-Cas system, configured to knockout or at least partially silence both alleles of a target endogenous gene; and a nucleic acid encoding an exogenous functional version of the target endogenous gene.

In some embodiments, the CRISPR-Cas system comprising at least one Cas protein and at least one gRNA, wherein each gRNA is configured to hybridize to a portion of the nucleic acid sequence encoding the target endogenous gene. In some embodiments, the at least one gRNA is configured to not hybridize to the exogenous functional version of the target endogenous gene. In some embodiments, the exogenous functional version of the target endogenous gene comprises a nucleic acid sequence different from that of the target endogenous gene and encodes for a polypeptide comprising an amino acid sequence at least 90% identical to that of the wild-type version of the target endogenous gene.

In some embodiments, the one or more nucleic acids comprises one or more messenger RNAs, one or more vectors, or a combination thereof.

In some embodiments, the CRISPR-Cas system is a CRISPR interference (CRISPRi) system. In some embodiments, the CRISPRi system comprises: at least one Cas protein; at least one gRNA, wherein each gRNA is configured to hybridize to a portion of the nucleic acid sequence encoding the target endogenous gene; and a transcriptional repressor. In some embodiments, the Cas protein and the transcriptional repressor are provided as a fusion protein, or a nucleic acid encoding thereof In some embodiments, the transcriptional repressor and the Cas protein or the at least one gRNA each comprise one half of a binding pair from a recruitment system. In some embodiments, the Cas protein is catalytically dead. In some embodiments, the Cas protein is Cas9, Cas12a, and Cas14.

In some embodiments, the Cas protein, the at least one gRNA, and when included, the transcriptional repressor are provided on a single nucleic acid. In some embodiments, the single nucleic acid is a vector. In some embodiments, the single nucleic acid is a baculovirus or lentivirus vector.

In some embodiments, the target endogenous gene is a disease-associated gene. In some embodiments, at least one allele of the target endogenous gene has a gain of function mutation. In some embodiments, at least one allele of the target endogenous gene has a loss of function mutation. In some embodiments, the target endogenous gene is BEST1. In some embodiments, the BEST1 gene comprises a D203A, I205T or Y236C mutation.

Further provided herein are methods of delaying the onset of, treating, preventing and/or curing a neurodegenerative disease in a subject in need thereof, comprising administering to the subject an effective amount of one or more viral vectors comprising a CRISPR system, wherein the CRISPR system silences both alleles of an endogenous gene and a nucleic acid encoding the wild type allele of the endogenous gene. Further provided herein are method of restoring gene function, comprising administering an effective amount of one or more viral vectors comprising a CRISPR system, wherein the CRISPR system silences both alleles of an endogenous gene and a nucleic acid encoding the wild type allele of the endogenous gene.

In some embodiments, the gene is BEST1. In some embodiments, the subject has a gain of function mutation in the endogenous gene. In some embodiments, the subject has a loss of function mutation in the endogenous gene.

In some embodiments, the viral vector is baculovirus or lentivirus. In some embodiments, the gain of function mutation is D203A, I205T or Y236C. In some embodiments, the viral vector comprising the CRISPR system comprises a baculovirus-based silencing (BVSi) vector containing a CMV promoter driven dCas9-KRAB-MeCP2-T2A-GFP expression cassette and a U6 promoter driven gRNA expression cassette.

In some embodiments, the CRISPR system targets exon 3 of the BEST1 gene and the gRNA has the sequence CTCACCCAGCACGAAGGAAA (SEQ ID NO: 1).

In some embodiments, the viral vector comprising the nucleic acid encoding the wild type allele baculovirus comprises a wobble WT BEST1-mCherry resistant to the recognition by gRNA of the CRISPR system.

Other aspects and embodiments of the disclosure will be apparent in light of the following detailed description and accompanying figures.

### BRIEF DESCRIPTION OF THE DRAWINGS

FIGS. 1A-1H show the functional influence of BEST1 loss-of-function mutants in HEK293 cells. FIG. 1A is a graph of the population steady-state current density-voltage relationships in HEK293 cells expressing BEST1 WT-CFP only (black), WT-CFP: WT-YFP=1:1 (gray), or WT-CFP: WT-YFP =1:4 (light gray), in the presence of 1.2 μM [Ca²⁺]ᵢ, n=5-6 for each point. FIGS. 1B-1H are graphs of the population steady-state current density-voltage relationships in HEK293 cells expressing BEST1 WT-CFP: mutant-YFP=1:1 (cyan), WT-CFP: mutant-YFP=1:4 (magenta), compared to mutant (red) or WT (circles) only, in the presence of 1.2 μM [Ca²⁺]ᵢ, n=5-6 for each point. The mutants are BEST1 A10T (FIG. 1B), R218H (FIG. 1C), L234P (FIG. 1D), A243T (FIG. 1E), Q293K (FIG. 1F), D302A (FIG. 1G) and P274R (FIG. 1H). All error bars in this figure represent s.e.m.

FIGS. 2A-2D show the functional influence of BEST1 gain-of-function mutants in HEK293 cells. FIGS. 2A-2C, left, are graph of population steady-state current density-voltage relationships in HEK293 cells co-expressing WT-CFP: mutant-YFP=1:1 (cyan) compared to WT only (WT-CFP: WT-YFP=1:1, gray), in the absence (open) or presence (solid) of 1.2 μM [Ca²⁺]ᵢ, n=5-6 for each point. FIGS. 2A-2C, right, are graphs of population steady-state current density-voltage relationships in HEK293 cells co-expressing WT-CFP: mutant-YFP=4:1 (blue) compared to mutant only (red), in the absence (open) or presence (solid) of 1.2 μM [Ca²⁺]ᵢ, n=5-6 for each point. The mutants are BEST1 D203A (FIG. 2A), I205T (FIG. 2B), and Y236C (FIG. 2C). All error bars in this figure represent s.e.m. WT or mutant BEST1-YFP-His was co-expressed with WT BEST1-CFP-Myc in HEK293 cells and detected by immunoblotting directly in cell lysate (input) or after co-immunoprecipitation (FIG. 2D).

FIGS. 3A-3E show BEST1 is responsible for conducting Ca²⁺-dependent Cl⁻ currents in hPSC-RPE. FIG. 3A is Ca²⁺-dependent Cl⁻ currents measured by whole-cell patch clamp in WT hPSC-RPE. Left, representative current traces recorded at 1.2 μM [Ca²⁺]ᵢ. Inset, voltage protocol used to elicit currents. Scale bar, 1 nA, 140 ms. Middle, population steady-state current density-voltage relationship in WT hPSC-RPE (black) compared to that from WT iPSC-RPE (gray), at 1.2 μM [Ca²⁺]ᵢ, n=5-6 for each point. Right, steady-state current density recorded at +100 mV plotted vs. free [Ca²⁺]ᵢ, from WT hPSC-RPE (black) compared to that from WT iPSC-RPE (gray), n=5-6 for each point. The plot was fitted to the Hill equation. FIGS. 3B-3E show Ca²⁺-dependent Cl⁻ currents measured by whole-cell patch clamp in BEST1^{-/-} (FIG. 3B), TMEM16A^{-/-} (FIG. 3C), TMEM16B^{-/-} (FIG. 3D), or LRRC8A^{-/-} (FIG. 3E) hPSC-RPE cells, respectively. Left, representative current traces recorded at 1.2 μM [Ca²⁺]ᵢ. Middle, population steady-state current density-voltage relationship in knockout hPSC-RPE cells (triangles), compared to that from WT hPSC-RPE cells (circles), at 1.2 μM [Ca²⁺]ᵢ, n=5-6 for each point. Right, steady-state current density recorded at +100 mV plotted vs. free [Ca²⁺]ᵢ from knockout (red) and WT BEST1 supplemented (triangles above Kd in FIG. 3B) hPSC-RPE cells, compared to the plot from WT hPSC-RPE (dotted black), n=5-6 for each point. Plots were fitted to the Hill equation. *P<0.05 compared to WT cells, using two-tailed unpaired Student t test. All error bars in this figure represent s.e.m.

FIGS. 4A-4F show Ca²⁺-dependent Cl⁻ currents in hPSC-RPE cells bearing BEST1 gain-of-function mutations. FIG. 4A is representative current traces of BEST1^{I205T/WT} hPSC-RPE in the absence of Ca²⁺. Scale bar, 1 nA, 140 ms. FIG. 4B is a graph of population steady-state current density-voltage relationships in BEST1^{I205T/WT} hPSC-RPE, in the absence (open red triangles) or presence (solid red triangles) of 1.2 μM [Ca²⁺]ᵢ, compared to cells with WT BEST1 augmentation in the absence of Ca²⁺ (open blue triangles), n=5-8 for each point. *P<0.05 compared to cells without augmentation in the absence of Ca²⁺, using two-tailed unpaired Student t test. FIG. 4C is a graph of steady-state current densities recorded at +100 mV plotted vs. free [Ca²⁺]ᵢ in BEST1^{I205T/WT} hPSC-RPE (triangles) compared to those in BEST1^{WT/WT} hPSC-RPE cells (circles), n=5-6 for each point. FIGS. 4D-4F show data for BEST1^{Y236C/WT} in the same format as FIGS. 4A-4C, respectively. *P<0.05 compared to cells without gene augmentation in the absence of Ca²⁺, using two-tailed unpaired Student t test. n=5-10 for each point. All error bars in this figure represent s.e.m.

FIGS. 5A-5F show knockdown and rescue of BEST1 gain-of-function mutations in hPCS-RPE cells. FIG. 5A is a graph of population steady-state current density-voltage relationships in WT hPSC-RPE cells treated with BVSi-Ctrl (black squares) compared to those in BVSi 3-8 (red circles) or BVSi 5-4 (blue triangle) treated cells, at 1.2 μM [Ca²⁺]ᵢ, n=5-17 for each point. *P<0.05 compared to BVSi-Ctrl treated cells, using two-tailed unpaired Student t test. FIG. 5B is a graph of population steady-state current density-voltage relationships in WT hPSC-RPE cells treated with BVSi 3-8 plus wobble WT BEST1 (grey) compared to those in untreated cells (black), at 1.2 μM [Ca²⁺]ᵢ, n=5-6 for each point. FIGS. 5C-5D are graphs of population steady-state current density-voltage relationships in BEST1^{I205T/WT} (FIG. 5C) or BEST1^{Y236C/WT} (FIG. 5D) hPSC-RPE cells treated with BVSi 3-8 alone (red circles), or BVSi 3-8 plus wobble WT BEST1 (blue triangles), at 1.2 μM [Ca²⁺]ᵢ, n=5-9 for each point. *P<0.05 compared to cells treated with BVSi 3-8 alone, using two-tailed unpaired Student t test. FIGS. 5E-5F are graphs of steady-state current densities recorded at +100 mV plotted vs. free [Ca²⁺]ᵢ in BEST1^{I205T/WT} (FIG. 5E) or BEST1^{Y236C/WT} (FIG. 5F) hPSC-RPE cells treated with BVSi 3-8 plus wobble WT BEST1 (blue triangles) compared to those in untreated WT hPSC-RPE (black dots), n=5-6 for each point. The plots were fitted to the Hill equation. All error bars in this figure represent s.e.m.

FIGS. 6A and 6B show electrophysiological analysis of BEST1 loss-of-function mutations. FIG. 6A is a bar chart showing population steady-state current densities at +100 mV for 1:1 co-expressed BEST1 WT-CFP and WT/mutant-YFP in HEK293 cells at 1.2 μM [Ca²⁺]ᵢ; n=5-6 for each point. FIG. 6B Bar chart showing population steady-state current densities at +100 mV for 1:4 co-expressed BEST1 WT-CFP and WT/mutant-YFP in HEK293 cells at 1.2 μM [Ca²⁺]ᵢ; n=5-6 for each point. *P<0.05 compared to WT only, using two-tailed unpaired Student t test. All error bars in this figure represent s.e.m.

FIGS. 7A and 7B show Electrophysiological analysis of BEST1 gain-of-function mutations. FIG. 7A is a bar chart showing population steady-state current densities at +100 mV for 1:1 co-expressed BEST1 WT-CFP and WT/mutant-YFP in HEK293 cells in the absence (open) or presence (solid) of 1.2 μM [Ca²⁺]ᵢ, n=5-6 for each point. *P<0.05 compared to WT only in the absence or presence of Ca²⁺, respectively, using two-tailed unpaired Student t test. FIG. 7B is a bar chart showing population steady-state current densities at +100 mV for 4:1 co-expressed BEST1 WT-CFP and WT/mutant-YFP in HEK293 cells in the absence (open) or presence (solid) of 1.2 μM [Ca²⁺]ᵢ; n=5-6 for each point. *P<0.05 compared to WT only in the absence or presence of Ca²⁺, respectively, using two-tailed unpaired Student t test. All error bars in this figure represent s.e.m.

FIGS. 8A and 8B are Western blots showing the expression of RPE-specific proteins BEST1, RPE65, CRALBP, and the loading control β-Action in hPSC-RPE (FIG. 8A) and iPSC-RPE (FIG. 8B) cells. Two gels/blots were prepared from the same cell lysate of each PSC-RPE to detect BEST1+β-Actin, and RPE65+CRALBP, respectively.

FIGS. 9A-9C show mRNA levels of CaCCs in hPSC-RPE cells. FIG. 9A is RT-PCR detecting BEST1 and control β-Action mRNA in WT native RPE, iPSC-RPE and hPSC-RPE cells. Plasmids bearing the corresponding full length cDNA were used as positive controls in PCR reactions. FIG. 9B is RT-PCR detecting mRNA of TMEM16A, TMEM16B, and LRRC8A in WT native RPE, iPSC-RPE and hPSC-RPE cells. Plasmids bearing the corresponding full length cDNA were used as positive controls in PCR reactions. FIG. 9C is RT-PCR detecting BEST1 and control β-Action mRNA in WT and knockout hPSC-RPE cells.

FIGS. 10A-10D show Ca²⁺-dependent Cl⁻ currents in iPSC-RPE and hPSC-RPE cells. FIG. 10A is Ca²⁺-dependent Cl⁻ currents measured by whole-cell patch clamp in patient-derived BEST1 null iPSC-RPE. Representative current traces recorded at 1.2 μM [Ca²⁺]ᵢ. Scale bar, 1 nA, 140 ms. FIG. 10B is a graph of population steady-state current density-voltage relationship in BEST1 null iPSC-RPE (red triangles) compared to that in WT iPSC-RPE (gray circles), at 1.2 μm [Ca²⁺]ᵢ, n=5-6 for each point. *P<0.05 compared to WT cells, using two-tailed unpaired Student t test. FIG. 10C is a graph of steady-state current density recorded at +100 mV plotted vs. free [Ca²⁺]ᵢ from BEST1 null (red triangles), and BEST1 null supplemented with WT BEST1 (blue triangles), compared to WT iPSC-RPE (gray), n=5-6 for each point. The plots were fitted to the Hill. FIG. 10D is a bar chart showing population steady-state current densities at +100 mV in hPSC-RPE cells at 1.2 μM [Ca²⁺]ᵢ, n=5-6 for each point. Two clonal hPSC-RPE cells from each genotype were recorded. Black, WT. Gray, knockout or knock-in mutants. All error bars in this figure represent s.e.m.

FIGS. 11A-11D show CRISPR/Cas9-mediated gene silencing in combination with augmentation. Augmented BEST1-GFP and endogenous BEST1 were detected by immunoblotting in hPSC-RPE cells (FIG. 11A). FIG. 11B is a schematic of the BVSi vector. FIG. 11C is Western blots showing the knockdown of endogenous BEST1 expression with BVSi vectors and augmentation of wobble BEST1-mCherry in WT hPCS-RPE cells. FIG. 11D is Western blots showing the knockdown of endogenous BEST1 expression with BVSi 3-8 and augmentation of wobble BEST1-mCherry in hPCS-RPE cells carrying BEST1 gain-of-function mutations.

FIGS. 12A and 12B show patient-derived BEST1 mutations in a homology model. Ribbon diagram of oppositely facing (144°) protomers of a BEST1 pentamer are shown with the extracellular side on the top. In FIG. 12A, the positions of the neck and the aperture are illustrated. In FIG. 12B, the side chains are shown and highlighted for residues harboring dominant (red) and recessive (blue) loss-of-function mutations, and gain-of-function mutations (magenta). In FIG. 12C, the side chains are shown and highlighted for residues at the neck (I76, F80 and F84, salmon) and aperture (I205, magenta).

FIGS. 13A and 13B are uncropped blots of FIG. 2D and FIG. 8, respectively.

### DETAILED DESCRIPTION OF THE INVENTION

The present disclosure provides methods for rescuing gene function and the treatment and prevention of a disease or disorder (e.g., bestrophinopathies). As shown herein, a combo approach to rescue BEST1 gain-of-function mutations was developed; the endogenous BEST1 gene, including both the mutant and WT alleles, was suppressed by CRISPR/Cas9-mediated gene silencing, while an exogenous copy of functional BEST1 gene was simultaneously augmented. For gene silencing, a programmable transcriptional repressor composed of a nuclease-dead Cas9 (dCas9) fused with a bipartite KRAB-MeCP2 repressor domain in the C-terminus (dCas9-KRABMeCP2) was employed.

For the simultaneous delivery of the complete CRISPR machinery, a baculovirus-based silencing (BVSi) vector containing a CMV promoter driven dCas9-KRAB-MeCP2-T2A-GFP expression cassette and a U6 promoter driven BEST1-specific gRNA expression cassette was constructed. For augmentation of BEST1 in the presence of BVSi, a baculovirus bearing a wobble WT BEST1-mcherry resistant to the recognition by the gRNA was generated. When RPE cells bearing a BEST1 gain-of-function mutation were simultaneously infected with BVSi and wobble WT BEST1-mcherry baculoviruses, the cellular function of BEST1 was restored.

The functional influence of different classes of patient-derived mutations on the channel when the mutant and WT BEST1 were co-expressed at various ratios in HEK293 cells was also determined. Strikingly, the six autosomal dominant loss-of-function mutations all behaved recessively at a 1:1 ratio with the WT BEST1, and required a 4:1 ratio to exhibit the mutant phenotype, suggestive of that they act in a dominant-negative manner rather than the canonical dominant manner, which clarifies results that gene augmentation is sufficient for the rescue of autosomal dominant loss-of-function mutations. Consistent with this finding, the mutant BEST1 allele was transcribed at a higher level than the WT allele in patient-derived RPE cells. In sharp contrast, the three autosomal dominant gain-of-function mutations all displayed a dominant behavior, even at an inferior 1:4 ratio with the WT BEST1. Due to their strong dominant effect, BEST1 gain-of-function mutations cannot be rescued by gene augmentation alone, but require CRISPR/Cas9-mediated silencing of the endogenous BEST1 in combination with gene augmentation for restoring Ca²⁺-dependent Cl⁻ currents in RPE cells. Additionally, the physiological role of BEST1 as the bona fide Ca2+-dependent Cl− current (CaCC) was confirmed in RPE. Taken together, the results reveal the differences between loss- and gain-of-function mutations, and provide a therapeutic strategy for all BEST1 mutations.

As disclosed herein BEST1 patient-derived loss-of-function and gain-of-function mutations require different mutant:wild-type (WT) molecule ratios for phenotypic manifestation, underlying their distinct epigenetic requirements in bestrophinopathy development, and suggesting that some of the previously defined autosomal dominant mutations actually behave in a dominant-negative manner. Importantly, the strong dominant effect of BEST1 gain-of-function mutations prohibits the restoration of BEST1-dependent Cl⁻ currents in RPE cells by gene augmentation, in contrast to the efficient rescue of loss-of-function mutations via the same approach. Gain-of-function mutations were rescuable by a combination of gene augmentation with CRISPR/Cas9-mediated knockdown of endogenous BEST1 expression, providing a universal treatment strategy for all bestrophinopathy patients regardless of their mutation types.

Thus, provided herein are therapeutic methods for the treatment and prevention of disease and disorders, e.g., ocular or neurodegenerative diseases, e.g., those associated with gain-of-function mutations. Also provided herein are therapeutic methods for the treatment and prevention of bestrophinopathies associated with BEST1 gain-of-function mutations, which are also suitable for loss-of-function mutations.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, molecular biology, immunology, and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing,” are used interchangeably herein and refer to the placement into a subject by a method or route which results in at least partial localization a desired site. Administration can be by any appropriate route which results in delivery to a desired location in the subject.

As used herein, “CRISPR-Cas system” refers collectively to transcripts and other elements involved in the expression of and/or directing the activity of CRISPR-associated (“Cas”) genes, including sequences encoding a Cas gene, Cas protein, a cr (CRISPR) sequence (e.g., crRNA or an active partial crRNA), or other sequences and transcripts from a CRISPR locus. In some embodiments, one or more elements of a CRISPR system is derived from a type I, type II, or type III CRISPR system.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, a “nucleic acid” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. The term “nucleic acid” may also encompass a chain comprising non-natural nucleotides, modified nucleotides, and/or non-nucleotide building blocks that can exhibit the same function as natural nucleotides (e.g., “nucleotide analogs”). Further, the term “nucleic acid” as used herein refers to an oligonucleotide, nucleotide or polynucleotide, and fragments or portions thereof, and to DNA or RNA of genomic or synthetic origin, which may be single or double-stranded, and represent the sense or anti sense strand.

As used herein, “percent sequence identity,” “percent identity,” and the like refer to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

A “polypeptide,” “protein,” or “peptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide,” “protein,” and “peptide” are used interchangeably herein.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or devices described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of devices and systems contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods

The present disclosure provides methods for restoring functionality of a target endogenous gene in a cell. The present disclosure also provides methods for delaying the onset of, treating, preventing and/or curing a disease or disorder in a subject.

In some embodiments, the methods comprise introducing into a cell: an effective amount of a Clustered Regularly Interspaced Short Palindromic Repeats (CRISPR)-Cas system, or one or more nucleic acids encoding the CRISPR-Cas system, configured to knockout or at least partially silence both alleles of a target endogenous gene; and a nucleic acid encoding an exogenous functional version of the target endogenous gene.

In some embodiments, the target endogenous gene is a disease associated gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. In some embodiments, at least one allele of the target endogenous gene has a gain of function mutation. In some embodiments, at least one allele of the target endogenous gene has a loss of function mutation.

Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM) (ncbi.nim.nih.gov/entrez/query.fcgi?db=OMIM); and the Human Gene Mutation Database (HGMD) (hgmd.cfac.uk).

Diseases caused by the contribution of multiple genes which lack simple (e.g., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. Thus, in some embodiments, the function of more than one target endogenous gene (e.g., 2, 3, 4, or more) can be restored using the disclosed methods.

In some embodiments, the target endogenous gene is BEST1. In some embodiments, at least one allele of BEST1 has a gain of function mutation. In some embodiments, at least one allele of BEST1 has a loss of function mutation. In some embodiments, the BEST1 comprises a D203A, I205T or Y236C mutation.

In some embodiments, the cell is a eukaryotic cell. In some embodiments, the cell is a mammalian cell. In some embodiments, the cell is a human cell. In some embodiments, the cell is in vitro. In some embodiments, the cell is in vivo. In some embodiments, introducing into a cell comprises administering to a subject.

In some embodiments, the subject has or is suspected to have a neurodegenerative disease. In some embodiments, the subject has or is suspected to have an ocular disease. Thus, in some embodiments, the disclosed methods can be used to delay the onset of, treat, prevent and/or cure an ocular disease or a neurodegenerative disease in a subject.

In some embodiments, the disease or disorder is a bestrophinopathy. Bestrophinopathies are a group of five retinal degeneration disorders caused by genetic mutations in the human BEST1 gene, namely best vitelliform macular dystrophy (BVMD), autosomal recessive bestrophinopathy (ARB), adult-onset vitelliform dystrophy (AVMD), autosomal dominant vitreoretinochoroidopathy (ADVIRC), and retinitis pigmentosa (RP). Clinical phenotypes of bestrophinopathies include serous retinal detachment, lesions that resemble egg yolk, or vitelliform, and progressive vision loss that can potentially lead to blindness. However, over 250 distinct BEST1 mutations have been identified from bestrophinopathy patients. The majority of the BEST1 mutations are autosomal dominant, whereas the autosomal recessive ones are mostly linked to ARB.

Functionally, Bestrophin-1 (BEST1), the protein encoded by BEST1, is a Ca²⁺-activated Cl⁻ channel (CaCC) predominantly expressed in retinal pigmented epithelium (RPE). Bestrophinopathy patient-derived RPE cells exhibited abnormal Ca²⁺-dependent Cl⁻ currents, underscoring the indispensable role of BEST1 as a CaCC in RPE, although the contribution of other candidate CaCCs cannot be excluded. Structurally, while the human BEST1 structure has not been solved, high resolution structures of three homologs from Klebsiella pneumoniae (KpBEST), chicken (cBEST1) and bovine (bBEST2) indicate that the channel is a highly conserved pentamer with a flower vase-shaped ion conducting pathway.

How each BEST1 mutation specifically affects the channel activity bestrophinopathy, eventually resulting in retinal degeneration, is largely unknown. The vast majority of the tested patient-derived mutations exhibited a loss-of-function phenotype, as the Cl⁻ currents mediated by the mutant channels are significantly reduced compared to the WT BEST1. Several gain-of-function mutations enhance the channel activity when transiently expressed in HEK293 cells but still cause bestrophinopathy, suggesting the physiological importance of maintaining normal BEST1 function. However, although most loss-of-function and all gain-of-function mutations known so far are autosomal dominant, whether they have different capacities to influence the channel activity at the presence of WT BEST1, as one would expect in a heterozygous carrier, remains elusive. In general, gain-of-function mutations often display a stronger dominant effect than loss-of-function mutations, but a side-by-side comparison between them has not been conducted for BEST1.

CRISPR-Cas systems have been successfully utilized to edit the genomes of various organisms, including, but not limited to bacteria, humans, fruit flies, zebra fish and plants. The invention is not limited by the type of CRISPR-Cas proteins or systems utilized. CRISPR-Cas systems are currently grouped into two classes (1-2), six types (I-VI) and dozens of subtypes, depending on the signature and accessory genes that accompany the CRISPR array. The CRISPR-Cas system of the present methods and systems is not limited by class, type, or subtype. In some embodiments, the present system may be derived from a Class 1 (e.g., Type I, Type III, Type VI) or a Class 2 (e.g. Type II, Type V, or Type VI) CRISPR-Cas system.

In some embodiments, the CRISPR-Cas system can be used to delete nucleic acids from the target endogenous gene by cleaving the target endogenous gene and allowing the host cell to repair the cleaved sequence in the absence of an exogenously provided donor nucleic acid molecule. Deletion of a nucleic acid sequence in this manner can be used to, for example, create gene knock-outs or knock-downs.

In some embodiments, the CRISPR-Cas system comprises a CRISPR interference (CRISPRi) system, and/or one or more nucleic acids encoding thereof. Thus, use of the CRISPR interference (CRISPRi) system, allows silencing of a gene through repression of transcription, usually by blocking either transcriptional initiation or elongation.

In some embodiments, CRISPRi system comprises at least one or all of: a guide RNA (gRNA) configured to hybridize to a portion of the nucleic acid sequence encoding the target endogenous gene; a Cas protein; and a transcriptional repressor. In some embodiments, the at least one gRNA, the Cas protein, and the transcriptional repressor are provided on a single nucleic acid. In some embodiments, the at least one gRNA, the Cas protein, and the transcriptional repressor are provided more than one nucleic acid.

Cas proteins are described in further detail in, e.g., Haft et al., PLoS Comput. Biol., 1(6): e60 (2005), incorporated herein by reference. The Cas protein may be any Cas endonucleases. In some embodiments, the Cas endonuclease is a Class 2 Cas endonuclease. In some embodiments, the Cas endonuclease is a Type V Cas endonuclease. In some embodiments, the Cas protein is Cas9, Cas12a, otherwise referred to as Cpf1, and Cas14.

In one embodiment, the Cas9 protein is a wild-type Cas9 protein. The Cas9 protein can be obtained from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas protein is a catalytically dead Cas, such as dCas9, dCas12a/dCpf1, dCas14, dead Cascade complex, or others. For example, catalytically dead Cas9 is essentially a DNA-binding protein due to, typically, two or more mutations within its catalytic nuclease domains which renders the protein with very little or no catalytic nuclease activity. Streptococcus pyogenes Cas9 may be rendered catalytically dead by mutations of D10 and at least one of E762, H840, N854, N863, or D986, typically H840 and/or N863 (see, e.g., U.S. Patent Application Publication 2017/0051312, incorporated herein by reference). Mutations in corresponding orthologs are known, such as N580 in Staphylococcus aureus Cas9. Oftentimes, such mutations cause catalytically dead Cas proteins to possess no more than 3% of the normal nuclease activity.

The guide RNA (gRNA) may be a crRNA, crRNA/tracrRNA (or single guide RNA, sgRNA). The gRNA may be a non-naturally occurring gRNA. The terms “gRNA,” “guide RNA” and “guide sequence” may be used interchangeably throughout and refer to a nucleic acid comprising a sequence that determines the binding specificity of the Cas protein. A gRNA hybridizes to, and/or is complementary to, partially or completely, a portion of the target endogenous gene.

The gRNA or portion thereof that hybridizes to the target endogenous gene (a target site) may be any length necessary for selective hybridization. gRNAs or sgRNA(s) can be between about 5 and about 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In some embodiments, two or more gRNA (e.g., 2, 3, 4, 5, 6, 7, 8, 9, 10, or more) are directed to each target endogenous gene. The gRNA may be a non-naturally occurring gRNA.

As used herein, the term “transcriptional repressor” refers to a protein or polypeptide that interacts with, directly or indirectly, specific DNA sequences associated with a genomic locus or gene of interest to prevent RNA polymerase activity to the promoter site for a gene or set of genes. Transcriptional repressors can be either mammalian cellular endogenous proteins that have repressor function, repressors from other species such as viruses, microbials or plants, their partial or mutant variants, engineered repressors, or other forms of repressor that can decrease gene expression. Exemplary repressors include but are not limited to: those with a Kruppel associated box (KRAB) domain (e.g., KOX1/ZNF10, KOX8/ZNF708, ZNF43, ZNF184, ZNF91, HPF4, HTF10 and HTF34), lac repressor, tryptophan repressor, and RE-1 silencing transcription factor (REST))

In some embodiments, the transcriptional repressor is fused to the Cas protein. In some embodiments, the transcriptional repressor comprises two or more transcription effector domains (e.g., transcriptional repressor domains) fused to the Cas protein. The two or more effector domains can be fused to the Cas protein in any orientation and may be separated from each other with an amino acid linker.

In some embodiments, the transcriptional repressor, and the Cas protein each comprise one half of a binding pair from a recruitment system. In some embodiments, the transcriptional repressor and the at least one gRNA each comprise one half of a binding pair from a recruitment system.

The recruitment system can comprise any two binding pairs. For example, the recruitment system may comprise an aptamer and an aptamer binding protein. In some embodiments, the aptamer sequence is a nucleic acid (e.g., RNA aptamer) sequence. In some embodiments, the guide RNA also comprises a sequence of one or more RNA aptamers, or distinct RNA secondary structures or sequences that can recruit and bind another molecular species, an adaptor molecule, such as a nucleic acid or protein. Any RNA aptamer/aptamer binding protein pair known may be selected and used in connection with the present disclosure (see, e.g., Jayasena, S. D., Clinical Chemistry, 1999. 45(9): p. 1628-1650; Gelinas, et al., Current Opinion in Structural Biology, 2016. 36: p. 122-132; and Hasegawa, H., Molecules, 2016; 21(4): p. 421, incorporated herein by reference).

In some embodiments, the aptamer sequence is a peptide aptamer sequence. In some embodiments, the Cas protein comprises the aptamer sequence and the transcriptional repressor comprises the aptamer binding protein. In some embodiments, the transcriptional repressor comprises the aptamer sequence and the Cas protein comprises the aptamer binding protein. The peptide aptamer sequence or aptamer binding protein may be fused in any orientation (e.g., N-terminus to C-terminus, C-terminus to N-terminus, N-terminus to N-terminus). The peptide aptamer sequence or aptamer binding protein may be fused by a linker region. Suitable linker regions are known in the art. The linker may be flexible or configured to allow the functionality and association with the DNA or other proteins with decreased steric hindrance. The linker sequences may provide an unstructured or linear region of the polypeptide, for example, with the inclusion of one or more glycine and/or serine residues. The linker sequences can be at least about 2, 3, 4, 5, 6, 7, 8, 9, 10 or more amino acids in length.

The peptide aptamers can be naturally occurring or synthetic peptides that are specifically recognized by an affinity agent. Such aptamers include, but are not limited to, a c-Myc affinity tag, an HA affinity tag, a His affinity tag, an S affinity tag, a methionine-His affinity tag, an RGD-His affinity tag, a 7× His tag, a FLAG octapeptide, a strep tag or strep tag II, a V5 tag, or a VSV-G epitope. Corresponding aptamer binding proteins are well-known in the art and include, for example, primary antibodies, biotin, affimers, single domain antibodies, and antibody mimetics.

To allow effective expression of the exogenous functional version of the target endogenous gene, the exogenous functional version of the target gene is not a target of the CRISPR-Cas system. In some embodiments, the at least one gRNA may target endogenous gene regions or mutations not found in the exogenous functional version. In some embodiments, the at least one gRNA is configured to not hybridize to the exogenous functional version of the target endogenous gene.

Alternatively, or in addition, the exogenous functional version of the target endogenous gene may comprise a nucleic acid sequence different from that of the target endogenous gene. In some embodiments, the exogenous functional version of the target endogenous gene comprises 5 or more (e.g., more than 5, more than 10, more that 15, more than 20, more than 25, more than 30, more than 35, more than 40, more than 45, more than 50) nucleotide changes from that of the endogenous gene sequence(s). In some embodiments, exogenous functional version of the target endogenous gene and the target endogenous gene differ in sequence in the region of gRNA binding, the location of the disease-causing mutation, in regions unrelated to gRNA binding or mutations, or any combination thereof. In select embodiments, the exogenous functional version of the target endogenous gene and the target endogenous gene differ in sequence in the region of gRNA binding such that the gRNA will not bind in any sufficient quantity to the exogenous functional version of the target endogenous gene.

In some embodiments, the exogenous functional version of the target endogenous gene encodes for a polypeptide comprising an amino acid sequence at least 90% (at least 90%, at least 91%, at least 92%, at least 93%, at least 94%, at least 95%, at least 96%, at least 97%, at least 98%, or at least 99%) identical to that of the wild-type version of the target endogenous gene.

In some embodiments, the exogenous functional version of the target endogenous gene encodes for a functional variant of the target endogenous gene. The functional variant should retain greater than 50% of the activity of the wild-type gene product. Electrophysiology assays may be used to readily determine functional variants of interest.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′-and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Thus, the disclosure further provides for cells comprising a sequence specific transcriptional activation system as disclosed herein, a nucleic acid, or a vector, as disclosed herein.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated, baculoviral, and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant baculoviruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan 1;459(1-2):70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines.

Methods for selecting suitable mammalian cells and methods for transformation, culture, amplification, screening, and purification of cells are known in the art.

In some embodiments, the cell is a eukaryotic cell. In some embodiments, the cell is a mammalian cell. In some embodiments, the cell is a human cell. In some embodiments, the cell is in vitro. In some embodiments, the cell is ex vivo.

An “effective amount” refers to an amount sufficient to elicit a desired biological response (e.g., treating a condition). As will be appreciated by those skilled in the art, the effective amount may vary depending on such factors as the desired biological endpoint, pharmacokinetics, the condition being treated, the mode of administration, and the age and health of the subject. An effective amount encompasses therapeutic and prophylactic treatment.

A “therapeutically effective amount” is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount means an amount, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

In the methods disclosed herein, administration may be by any of those methods known in the art that facilitate administration systemically/peripherally or at the site of desired action, including but not limited to, oral (e.g., by ingestion); topical (including e.g. transdermal, intranasal, ocular, buccal, and sublingual); pulmonary (e.g., by inhalation or insufflation therapy using, e.g., an aerosol, e.g., through mouth or nose); rectal; vaginal; parenteral (e.g., by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal injection); or by implant of a depot, for example, subcutaneously or intramuscularly. In some embodiments, the administration is topical, local ocular (e.g., subconjunctival, retrobulbar, intracameral, intravitreal), or systemic.

### 3. Systems

The present invention is also directed to compositions or systems comprising a Clustered Regularly Interspaced Short Palindromic Repeats (CRISPR)-Cas system, or one or more nucleic acids encoding the CRISPR-Cas system, configured to knockout or at least partially silence both alleles of a target endogenous gene and a nucleic acid encoding an exogenous functional version of the target endogenous gene. Descriptions provided for the CRISPR-Cas system, the target endogenous gene, and the nucleic acids elsewhere herein are applicable to the disclosed compositions and systems.

In some embodiments, the system comprises a CRISPR interference (CRISPRi) system, and/or one or more nucleic acids encoding thereof, configured to knockout or at least partially silence both alleles of a target endogenous gene. In some embodiments, the CRISPRi system comprises a transcriptional repressor.

In some embodiments, one or more all of the at least one gRNA, the Cas protein, and the transcriptional repressor are provided on a single nucleic acid. In some embodiments, each of the at least one gRNA, the Cas protein, and the transcriptional repressor are provided more than one nucleic acid.

In some embodiments, the Cas protein and the transcriptional repressor are provided as a fusion protein, as described above. In some embodiments, the transcriptional repressor, and the Cas protein or the at least one gRNA each comprise one half of a binding pair from a recruitment system, as described above.

### 4. Kits

Also within the scope of the present disclosure are kits including at least one or all of the components of the CRISPR-Cas system (e.g., a guide, a Cas protein, a transcriptional repressor, and/or one or more nucleic acids encoding thereof) and/or the nucleic acid encoding an exogenous functional version of the target endogenous gene, a composition as described herein, or the vectors and materials (e.g., cells and delivery systems) which to facilitate production of any of the prior listed components.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may include instructions for use in any of the methods described herein.

The kits provided herein are in suitable packaging. Suitable packaging includes, but is not limited to, vials, bottles, jars, flexible packaging, and the like.

The present disclosure also provides for kits for performing the methods or producing the components in vitro. Optional components of the kit include one or more of the following: buffers, cell culture media or components thereof for use in generating the cells disclosed herein, nucleic acid sequences, sequencing primers, and the like.

### 5. Examples

### MATERIALS AND METHODS

Generation of human iPSC The CytoTune™-iPS 2.0 Sendai Reprogramming Kit (Thermo Fisher Scientific, A16517) was used to reprogram donor-provided skin fibroblasts into pluripotent stem cells (iPSC). Immunocytofluorescence assays were carried out following the previously published protocol to score iPSC pluripotency (Li, Y., et al., Methods Mol Biol 1353, 77-88, doi:10.1007/7651_2015_225 (2016), incorporated herein by reference in its entirety). The iPSC cells from all the subjects enrolled in this study were characterized by detecting four standard pluripotency markers (SSEA4, Tra-1-60, SOX2 and Nanog). Nuclei were detected by Hoechst staining. All iPSC lines were passaged every 3-6 days while maintained in mTeSR-1 medium (STEMCELL Technologies, 85850). The morphology and nuclear/cytoplasmic ratio were closely monitored to ensure the stability of the iPSC lines. All the iPSC lines were sent for karyotyping by G-banding to verify genome integrity at Cell Line Genetics (Wisconsin, USA).

Differentiation of iPSC and hPSC lines into RPE cells iPSC and hPSC lines were cultured to confluence in 6-well culture dishes pretreated with 1:50 diluted matrigel (CORNING, 356230). For the first 14 days, the differentiation medium consisted of Knock-Out (KO) DMEM (Thermo Fisher Scientific, 10829018), 15% KO serum replacement (Thermo Fisher Scientific, 10829028), 2 mM glutamine (Thermo Fisher Scientific, 35050061), 50 U/ml penicillin-streptomycin (Thermo Fisher Scientific, 10378016), 1% nonessential amino acids (Thermo Fisher Scientific, 11140050), and 10 mM nicotinamide (Sigma-Aldrich, N0636). During Day 15-28 of differentiation, the differentiation medium was supplemented with 100 ng/ml human Activin-A (PeproTech, 120-14). From Day 29 on, the differentiation medium without Activin-A supplementation was used again until differentiation was completed. After roughly 8-10 weeks, dispersed pigmented flatten clusters were formatted and manually picked to matrigel-coated dishes. These cells were kept in RPE culture medium as previously described (Maminishkis, A. et al. Investigative ophthalmology & visual science 47, 3612-3624, doi:10.1167/iovs.05-1622 (2006), incorporated herein by reference in its entirety). It takes another 6-8 weeks in culture for them to form a functional monolayer, which would be ready for function assays. In addition to well-established classical mature RPE markers (Bestrophinl, CRALBP and RPE65), two more markers (PAX6 and MITF) were also used to validate the RPE fate of the cells. All iPSC-RPE cells in this study were at passage 1. DNA sequencing was used to verify genomic mutations in the mutant iPSC-RPE cells.

Cell lines HEK293 cells were kindly gifted from Dr. Henry Colecraft at Columbia University. As HEK293 is on the International Cell Line Authentication Committee's list of commonly misidentified cell lines, the cells used in this study were authenticated by short tandem repeat (STR) DNA profiling and tested negative for mycoplasma contamination. The culture medium was DMEM (4.5 g/L glucose, Corning 10013CV) supplemented with 100 μg/ml penicillin-streptomycin and 10% fetal bovine serum. H1-iCas9 cells were purchased from the Stem Cell Research Facility of Memorial Sloan Kettering Cancer Center. The culture medium was mTeSR1 with supplement (STEMCELL Technologies, 85850).

Electrophysiology An EPC10 patch clamp amplifier (HEKA Electronics) controlled by Patchmaster (HEKA) was utilized to conduct whole-cell recordings 24-72 hours after splitting of RPE cells or transfection of HEK293 cells. Micropipettes were pulled and fashioned from 1.5 mm thin-walled glass with filament (WPI Instruments) and filled with internal solution containing (in mM): 130 CsCl, 10 EGTA, 1 MgCl₂, 2 MgATP (added fresh), 10 HEPES (pH 7.4, adjusted by CsOH), and CaCl₂ to obtain the desired free Ca²⁺ concentration (maxchelator.stanford.edu/CaMgATPEGTA-TS.htm). Series resistance was usually 1.5-2.5 Ma No electronic series resistance compensation was used. External solution contained (in mM): 140 NaCl, 15 glucose, 5 KC1, 2 CaCl₂, 1 MgCl₂ and 10 HEPES (pH 7.4, adjusted by NaOH). Solution osmolarity was between 310 and 315. A family of step potentials (−100 to +100 mV from a holding potential of 0 mV) were used to generate I-V curves. Currents were sampled at 25 kHz and filtered at 5 or 10 kHz. Traces were acquired at a repetition interval of 4 s. All experiments in this study were carried out at ambient temperature (23±2° C.).

Immunoblotting Cell pellets were extracted by the M-PER mammalian protein extraction reagent (Thermo Fisher Scientific, 78501) supplemented with proteinase inhibitors (Roche, 04693159001), and the protein concentration was quantified by a Bio-Rad protein reader. After denaturing at 95° C. for 5 min, the samples (20 μg) were run on 4-15% gradient SDS-PAGE gel at room temperature, and wet transferred onto nitrocellulose membrane at 4° C. The membranes were incubated in blocking buffer containing 5% (w/v) non-fat milk for 1 hour at room temperature, and subsequently incubated overnight at 4° C. in blocking buffer supplemented with primary antibody. Primary antibodies against the following proteins were used: CRALBP (1:500 Abcam, ab15051), RPE65 (1:1,000 Novus Biologicals, NB100-355), β-Actin (1:2,000 Abcam, ab8227), BEST1 (1:500 Novus Biologicals, NB300-164), His (1:1,000 Fisher Scientific, PA1983B) and Myc (1:1,000 Fisher Scientific, PA1981). Fluorophore-conjugated mouse and rabbit secondary antibodies (LI-COR Biosciences, 925-68070 and 925-32213, respectively) were used at a concentration of 1:10,000 and an incubation time of 1 h at room temperature, followed by infrared imaging.

Immunoprecipitation HEK293 cells cultured on 6-cm dishes were co-transfected with pBacMam-BEST1(WT)-CFP-Myc and pBacMam-BEST1(mutant or WT)-YFP-His at 1:1 ratio using PolyJet™ In Vitro DNA Transfection Reagent (SignaGen Laboratories, SL100688) following the manufacturer's standard manual. 48 h post transfection, cells were harvested by centrifugation at 1000×g for 5 min at room temperature. Cell pellets were lysed in pre-cooled lysis buffer (150 mM NaCl, 50 mM Tris, 0.5% IGEPAL® CA-630, pH 7.4) supplemented with protease inhibitor cocktails (Roche, 04693159001) for 30 min on ice, and then centrifuged at 13,000 rpm for 12 min at 4° C. The supernatant (300 μg) was collected and mixed with 2 μg Myc monoclonal antibody (Thermo Fisher Scientific, MA1-980). After rotating overnight at 4° C., the mixture was incubated with Dynabeads M-280 sheep anti-mouse IgG (Thermo Fisher Scientific, 11202D) for 5 h at 4° C. After thorough washing of the beads, bound fractions were eluted in lx SDS sample buffer (Biorad, 1610747) by heating for 10 min at 75° C. Proteins were then resolved by SDS-PAGE and analyzed by immunoblotting.

Baculovirus production and transduction BacMam baculovirus bearing BVSi 5-4-GFP, BVSi 3-8-GFP, BVSi-Ctrl-GFP, or wobble BEST1-mcherry were generated in-house as previously described (Goehring, A. et al., Nature protocols 9, 2574-2585, doi:10.1038/nprot.2014.173 (2014), incorporated herein by reference in its entirety). For transduction, the viruses were added to the culture medium of freshly split hPSC-RPE cells.

Molecular cloning Point mutations in BEST1 were made by site-directed mutagenesis PCR with the In-fusion Cloning Kit (Clontech). All constructs were fully sequenced.

Measuring allelic transcription level Total RNA was extracted from cell pellets with the PureLink RNA Mini Kit (ThermoFisher, 12183020) and subjected to cDNA synthesis using the RevertAid First Strand cDNA synthesis kit (ThermoFisher K1621). The resultant cDNA was used as the template for PCR amplification of the target BEST1 regions that contain mutations/polymorphisms, and the PCR products were sub-cloned using the TOPO Cloning Kit (ThermoFisher, 451245) for sequencing.

Knockout/knock-in in H1-iCas9 cells Doxycycline (2 μg/mL) was supplemented to the culture medium to induce Cas9 expression and maintained in the medium for 3 days. 24 h post doxycycline addition, the cells were transfected with gRNA (+ssDNA for knock-in) as previously described (Zhu, Z., et al., Methods in enzymology 546, 215-250, doi:10.1016/B978-0-12-801185-0.00011-8 (2014), incorporated herein by reference in its entirety). After recovery to ˜50% confluency, the cells were lifted by TrypLE (ThermoFisher, 12604013) treatment, and seeded to 2×10 cm fresh plates at 1,000 cells/plate and 2,000 cells/plate, respectively. 10-12 days later, single colonies became visible, and were picked into individual wells on a 96-well plate. After amplification, each single colony was subjected for genotyping by Sanger sequencing. For the knockout of BEST1, TMEM16A, TMEM16B and LRRC8A, gRNAs were designed to target N-terminal portion of the coding genomic sequences, such that all or most of the transmembrane domain is eliminated in the residual translated product (if exists), rendering functionally null.

gRNA design for CRISPR/Cas9-mediated gene editing/silencing The gRNAs were designed using online software (IDTdna.com) and are summarized in Table 1.

TABLE 1
gRNA sequences for CRISPR/Cas9

| | | Target- | | SEQ |
|---|---|---|---|---|
| | | ing | | ID |
| Gene | Purpose | Exon | gRNA sequence | NO: |
| TMEM16A | knockout | Exon 10 | CCATGGTGGCGCATCCGTAC | 2 |
| TMEM16B | knockout | Exon 13 | GCTGGCCTGCGCGGTCCCAC | 3 |
| LRRC8A | knockout | Exon 3 | TCCTTGGTGACCCACTTACA | 4 |
| BEST1 | knockout | Exon 3 | CTCACCCAGCACGAAGGAAA | 1 |
| | I205T | Exon 5 | TCTGGAGCAGGATAGGGTCC | 5 |
| | knock-in | | | |
| | Y236C | Exon 6 | TGTATACACAGGTGAGGACT | 6 |
| | knock-in | | | |
| | Silencing | Exon 3 | CTCACCCAGCACGAAGGAAA | 1 |
| | (BVSi 3-8) | | | |
| | Silencing | Exon 5 | GCAGGCTCTGGAGCAGGATA | 7 |
| | BVSi 5-4) | | | |

Transfection 20-24 h before transfection, HEK293 cells were lifted by incubation with 0.25% trypsin at room temperature for 5 min and split into new 3.5-cm culture dishes at proximately 50% confluency. Plasmids (1 μg) bearing the WT BEST1 or desired mutant were transfected using PolyJet transfection reagent (SignaGen SL100688). The transfection mix was removed after 6-8 h, and cells were rinsed with PBS once and cultured in supplemented DMEM. 24 h post transfection, cells were lifted again by trypsin treatment and split onto fibronectin-coated glass coverslips for patch clamp.

Electrophysiological data and statistical analyses Patch clamp data were analyzed off-line with Patchmaster (HEKA), Microsoft Excel and Origin. Statistical analyses were conducted using built-in functions in Origin. For comparisons between two groups, statistically significant differences between means (P<0.05) were determined using Student's t test. Data are presented as means±s.e.m.

Homology modeling of human BEST1 A homology model for BEST1 was generated using the Swiss-Model server from the chicken BEST1 crystal structure (See, Kane Dickson, V., et al., Nature 516, 213-218, doi:10.1038/nature13913 (2014)). The structural figure was made in PyMOL.

Human samples Skin biopsy samples were obtained from the healthy control donor and patient, and processed and cultured as previously described (Li, Y., et al., Methods Mol Biol 1353, 77-88, doi:10.1007/7651_2015_225 (2016), incorporated herein by reference in its entirety). For these procedures, all of which were approved by Columbia University Institutional Review Board (IRB) protocol AAAF1849, the donor and patient provided written informed consent. All methods were performed in accordance with the relevant regulations and guidelines. Donor native RPE was isolated from human autopsy eye shell which purchased from the Eye-Bank for Sight Restoration (New York, NY, 10005).

Key Resources Table

| Reagent type | | | | |
|---|---|---|---|---|
| (species) or | | Source or | | Additional |
| resource | Designation | reference | Identifiers | information |
| strain, strain | HST08 (Stellar | TaKaRa | 636766 | Chemical |
| background | cells) | | | competent cells |
| (Escherichia coli) | | | | |
| cell line | Sf9 | Thermo | RRID:CVC | Insect cell line |
| (Spodoptera | | Fisher | L_0549 | for baculovirus |
| frugiperda) | | Scientific | | production |
| cell line (Homo | HEK293 | ATCC | RRID:CVC | Embryonic |
| sapiens) | | | L_0045 | kidney cells |
| cell line (Homo | H1 iCas9 | Sloan | | Embryonic |
| sapiens) | | Kettering | | stem cell line |
| | | Institute, | | with an |
| | | González et | | inducible |
| | | al., 2014 | | CRISPR |
| | | | | cassette |
| cell line (Homo | H1 iCas9 | — | | BEST1 |
| sapiens) | BEST1^{-/-} | | | knockout of the |
| | | | | H1 iCas9 line |
| cell line (Homo | H1 iCas9 | — | | TMEM16A |
| sapiens) | TMEM16A⁻/- | | | knockout of the |
| | | | | H1 iCas9 line |
| cell line (Homo | H1 iCas9 | — | | TMEM16B |
| sapiens) | TMEM16B^{-/-} | | | knockout of the |
| | | | | H1 iCas9 line |
| cell line (Homo | H1 iCas9 | — | | LRRC8A |
| sapiens) | LRRC8A^{-/-} | | | knockout of the |
| | | | | H1 iCas9 line |
| cell line (Homo | H1 iCas9 | — | | BEST1^{I205T/WT} |
| sapiens) | BEST1^{I205T/WT} | | | knock-in of the |
| | | | | H1 iCas9 line |
| cell line (Homo | H1 iCas9 | — | | BEST1^{Y236C/WT} |
| sapiens) | BEST1^{Y236C/WT} | | | knock-in of the |
| | | | | H1 iCas9 line |
| biological sample | RPE cells | Li et al., 2017 | | Human RPE |
| (Homo sapiens) | | | | cells from a |
| | | | | post-mortem |
| | | | | donor |
| biological sample | iPSC-RPE cells | Ji et al., 2019 | | iPSC-RPE cells |
| (Homo sapiens) | | | | derived from |
| | | | | patient skin |
| | | | | cells |
| antibody | anti-RPE65 | Novus | Cat#: | WB (1:1,000) |
| | (Mouse | Biologicals | NB100-355, | |
| | monoclonal) | | RRID:AB_ | |
| | | | 10002148 | |
| antibody | anti-CRALBP | Abcam | Cat#: | WB (1:500) |
| | (Mouse | | ab15051, | |
| | monoclonal) | | RRID:AB_ | |
| | | | 2269474 | |
| antibody | anti-BEST1 | Novus | Cat#: | WB (1:500) |
| | (Mouse | Biologicals | NB300-164, | |
| | monoclonal) | | RRID:AB_ | |
| | | | 10003019 | |
| antibody | anti-ß-Actin | Abcam | Cat#: | WB (1:2,000) |
| | (Rabbit | | ab8227, | |
| | polyclonal) | | RRID:AB_ | |
| | | | 2305186 | |
| antibody | anti- 6xHis | Thermo | Cat#: PA1- | WB (1:1,000) |
| | (Rabbit | Fisher | 983B, | |
| | polyclonal) | Scientific | RRID:AB_ | |
| | | | 1069891 | |
| antibody | anti- Myc | Thermo | Cat#: PA1- | WB (1:1,000) |
| | (Rabbit | Fisher | 981, | |
| | polyclonal) | Scientific | RRID:AB_ | |
| | | | 325961 | |
| antibody | IRDye ® 680RD | LI-COR | Cat#: 925- | WB (1:10,000) |
| | goat anti-mouse | Biosciences | 68070, | |
| | IgG secondary | | RRID:AB_ | |
| | antibody | | 2651128 | |
| antibody | IRDye ® 800CW | LI-COR | Cat#: 925- | WB (1:10,000) |
| | donkey anti- | Biosciences | 32213, | |
| | Rabbit IgG | | RRID:AB_ | |
| | secondary | | 2715510 | |
| | antibody | | | |
| recombinant | pBacMam- | Li et al., 2017 | | To express |
| DNA reagent | BEST1-GFP | | | exogenous |
| | (plasmid) | | | BEST1 in |
| | | | | HEK293 cells |
| recombinant | pBacMam- | — | | To generate |
| DNA reagent | BEST1-mCherry | | | baculoviruses |
| | (plasmid) | | | for expressing |
| | | | | BEST1 in RPE |
| | | | | cells |
| recombinant | dCas9-KRAB- | Addgene | RRID | Improved |
| DNA reagent | MeCP2 | | :Addgene_1 | dCas9 |
| | (plasmid) | | 10821 | repressor- |
| | | | | dCas9-KRAB- |
| | | | | MeCP2 |
| recombinant | pSpCas9(BB)- | Addgene | RRID | Cas9 from S. |
| DNA reagent | 2A-GFP (PX458) | | :Addgene_4 | pyogenes with |
| | (plasmid) | | 8138 | 2A-EGFP, and |
| | | | | cloning |
| | | | | backbone for |
| | | | | sgRNA |
| recombinant | BVSi 5-4-GFP | — | | Baculoviral |
| DNA reagent | (plasmid) | | | construct for |
| | | | | Cas9-mediated |
| | | | | BEST1 |
| | | | | silencing |
| recombinant | BVSi 3-8-GFP | — | | Baculoviral |
| DNA reagent | (plasmid) | | | construct for |
| | | | | Cas9-mediated |
| | | | | BEST1 |
| | | | | silencing |
| recombinant | BVSi ctrl-GFP | — | | Baculoviral |
| DNA reagent | (plasmid) | | | construct for |
| | | | | the control of |
| | | | | Cas9-mediated |
| | | | | gene silencing |
| sequence-based | hBest1-I205T- | — | Knock-in | GCCCTGGGT |
| reagent | SSDNA | | ssDNA | GTGGTTTGC |
| | | | template | CAACCTGTC |
| | | | | AATGAAGGC |
| | | | | GTGGCTTGG |
| | | | | AGGTCGAAT |
| | | | | TCGGGACCC |
| | | | | TACCCTGCT |
| | | | | CCAGAGCCT |
| | | | | GCTGAACGT |
| | | | | GAGCCCACT |
| | | | | GTACAGACA |
| | | | | GGGCTGCCG |
| | | | | CAG (SEQ ID |
| | | | | NO: 8) |
| sequenced-based | hBest1-Y236C- | — | Knock-in | TCAGTGTGG |
| reagent | SSDNA | | ssDNA | ACACCTGTA |
| | | | template | TGCCTACGA |
| | | | | CTGGATTAG |
| | | | | TATCCCACT |
| | | | | GGTGTGTAC |
| | | | | ACAGGTGAG |
| | | | | GACTAGTCT |
| | | | | GGTGAGGCT |
| | | | | GCCCTTTTG |
| | | | | GGAAACTGA |
| | | | | GGCTAGAAG |
| | | | | GACCAAGG |
| | | | | AAGC (SEQ |
| | | | | ID NO: 9) |
| commercial assay | Cyto Tune TM-iPS | Thermo | Cat#: | To generate |
| or kit | 2.0 Sendai | Fisher | A16517 | iPSC |
| | reprogramming | Scientific | | |
| | kit | | | |
| commercial assay | In-Fusion HD | Clontech | Clontech:63 | For molecular |
| or kit | Cloning | | 9647 | cloning |
| commercial assay | PolyJetTM In | SignaGen | SL100688 | For cell |
| or kit | Vitro DNA | Laboratories | | transfection |
| | Transfection | | | |
| | Reagent | | | |
| software, | Patchmaster | HEKA | RRID:SCR | Patch clamp |
| algorithm | | | _000034 | data collection |
| | | | | and analysis |
| software, | PyMOL | PyMOL | RRID:SCR | Structural |
| algorithm | | | _000305 | analysis |

### EXAMPLE 1

### BEST1 Loss-of-Function Mutations Affect Cl⁻ Currents in a Dosage-Sensitive Manner

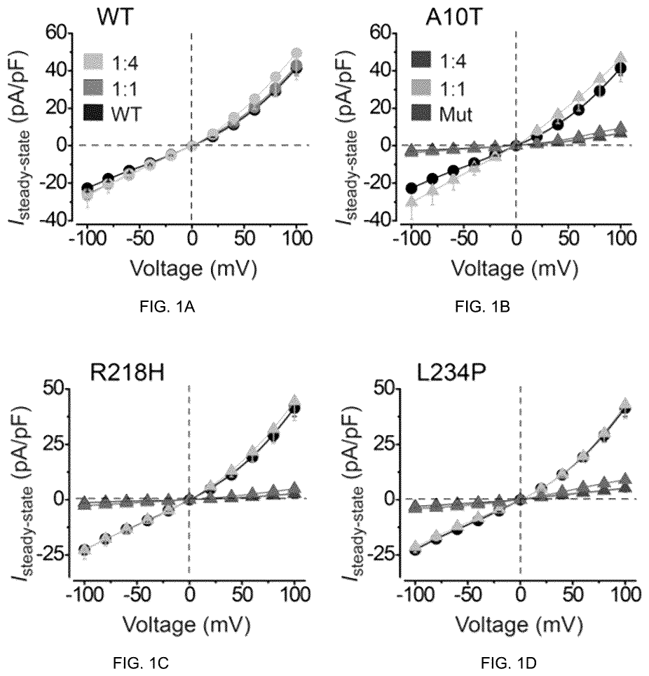

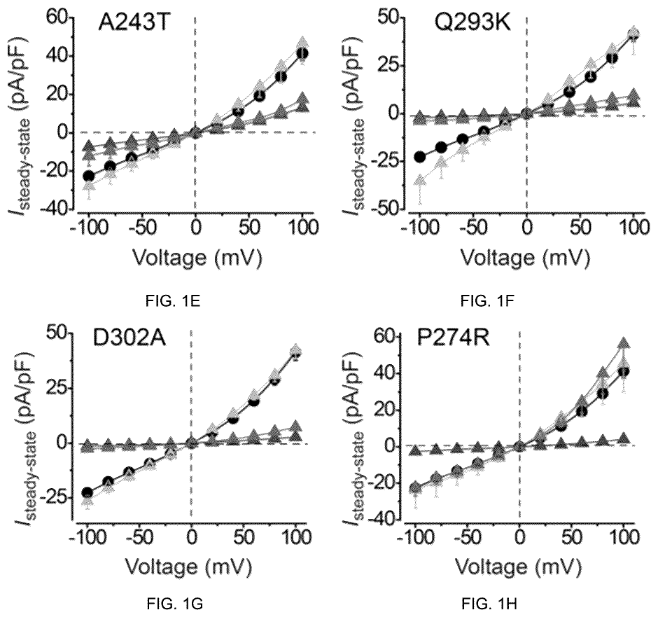

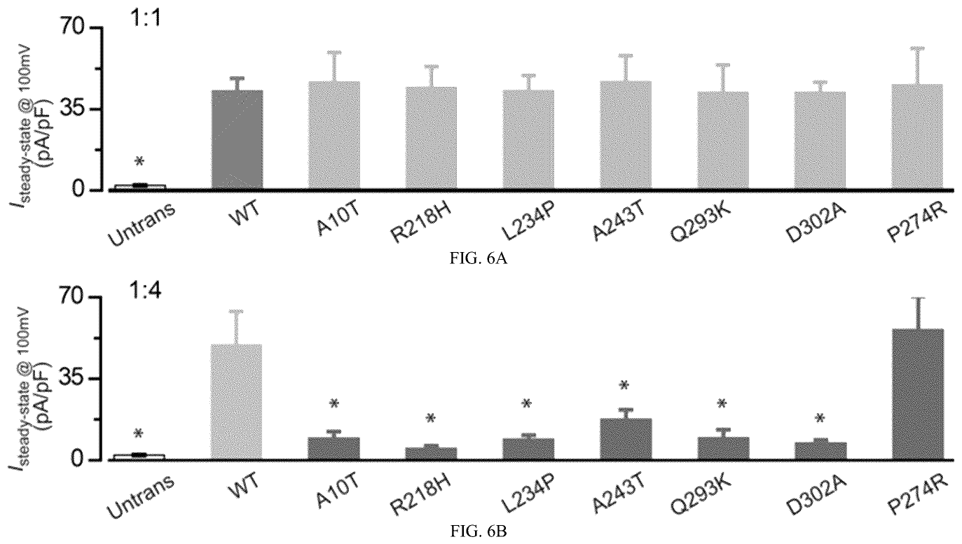

To quantitatively evaluate the influence of BEST1 mutations on the channel activity under a condition mimicking the endogenous gene dosage, seven YFP-tagged BEST1 loss-of-function mutants, including six autosomal dominant (A10T, R218H, L234P, A243T, Q293K and D302A) and one autosomal recessive (P274R), were individually mixed with CFP-tagged WT BEST1 at a 1:1 ratio and introduced into HEK293 cells for patch clamp recording. Surprisingly, in the presence of 1.2 μM free intracellular Ca²⁺ ([Ca²⁺]ᵢ), where BEST1 conducts peak current, Cl⁻ currents from cells co-expressing mutant and WT BEST1 were similar to those from cells expressing WT BEST1 alone (FIGS. 1A-1H and 6A), regardless of whether the mutation is autosomal dominant or recessive. Therefore, these six loss-of-function mutations, although genetically defined as autosomal dominant, do not exhibit dominant behavior in vitro.

To test if a dominant-negative effect is at play, the mutants were individually co-transfected with WT BEST1 at a 4:1 ratio into HEK293 cells for patch clamp analysis. At 1.2 μM [Ca²⁺]ᵢ, Cl⁻ currents from co-expression of an autosomal dominant mutant and WT BEST1 were significantly smaller than those from the WT only, and similar to those from the mutant only (FIGS. 1B-1G and 6B). By contrast, currents from cells co-expressing the WT BEST1 and the autosomal recessive P274R mutant were still similar to those from cells expressing the WT BEST1 only (FIGS. 1H and 6B). Therefore, the six previously recognized autosomal dominant mutations are actually dominant-negative in vitro, whereas the autosomal recessive P274R mutation indeed behaves recessively.

### EXAMPLE 2

### Imbalanced Transcription of BEST1 Alleles in Human RPE

The patch clamp results from transiently transfected HEK293 cells predict that the autosomal dominant mutant allele was expressed at a higher level than the WT allele in patients' RPE, such that the dominant-negative effect was manifested. To test this hypothesis, mRNA was extracted from patient-derived iPSC-differentiated RPE (iPSC-RPE), and the ratio of transcripts from the mutant and WT BEST1 alleles was measured by reverse transcription PCR (RT-PCR) and TOPO cloning. Remarkably, the mutant genotype showed up 3-4 times more than the WT in all 12 BVMD patient-derived iPSC-RPE clones (two clones from each patient) (Table 2), indicating that the transcription level of the mutant allele is 3-4 fold higher than that of the WT allele in these patients' RPE cells.

TABLE 2
Sequencing of BEST1 transcripts in RPE cells.

| | | RPE | Mutant/WT | Mutant/WT |
|---|---|---|---|---|
| Donor # | Mutation | type | from clonal #1 | from clonal #2 |
| 1 | A10T | iPSC-RPE | 72/23 | 51/12 |
| 2 | R218H | iPSC-RPE | 84/20 | 45/11 |
| 3 | L234P | iPSC-RPE | 77/19 | 42/20 |
| 4 | A243T | iPSC-RPE | 83/28 | 37/11 |
| 5 | Q293K | iPSC-RPE | 76/19 | 46/10 |
| 6 | D302A | iPSC-RPE | 78/18 | 35/14 |
| 7 | rs767552540 | Native | 74/23 | NA |

#1-6 are patient-derived iPSC-RPE cells carrying the same set of BEST1 mutations as those analyzed in transiently transfected HEK293 cells in FIG. 1.
#7 is native human RPE cells from a healthy donor bearing a SNP in the BEST1 gene.

To further validate if the two BEST1 alleles have imbalanced transcription in native RPE, RPE cells were collected from a post-mortem donor harboring heterozygosity of a single nucleotide polymorphism (rs767552540) in BEST1. Consistent with results from iPSC-RPE, transcripts from one allele outnumbered those from the other by ˜3 fold in these human native RPE cells (Table 2).

Together, the results suggested that allelic imbalance of BEST1 transcription contributes to the dominant-negative effect of the autosomal dominant mutations. Importantly, this provided an explanation for the restoration of Ca²⁺-dependent Cl⁻ currents by gene augmentation in iPSC-RPE cells bearing a BEST1 loss-of-function autosomal dominant mutation: as long as the augmented BEST1 WT protein was expressed at a similar or higher level compared to the endogenous BEST1, the mutant protein was no longer dominant-negative, such that the WT phenotype was exhibited as seen in transiently transfected HEK293 cells (FIGS. 1B-1G).

### EXAMPLE 3

### BEST1 Gain-of-Function Mutations are Bona Fide Dominant In Vitro

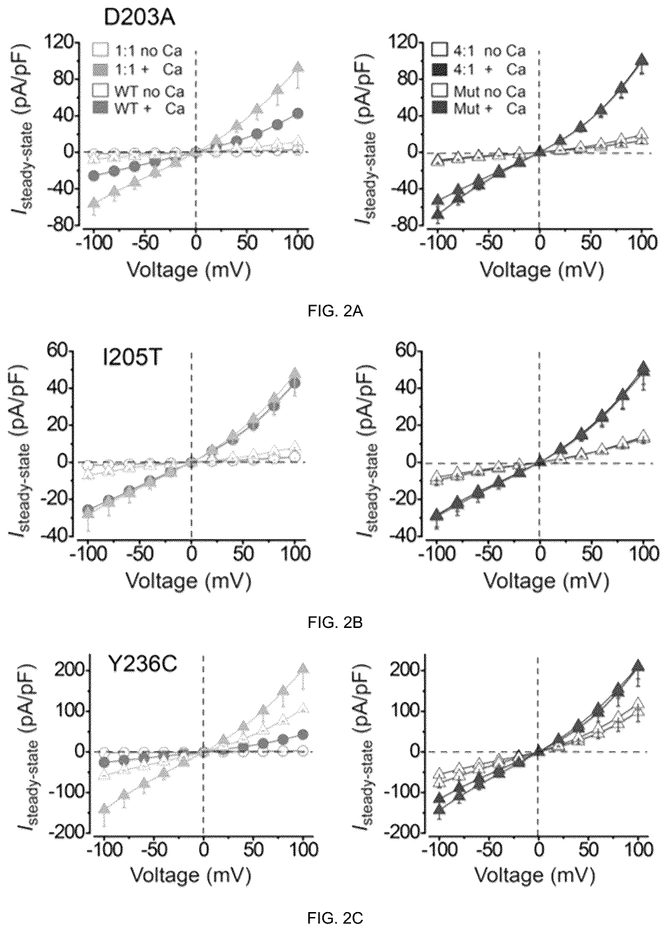

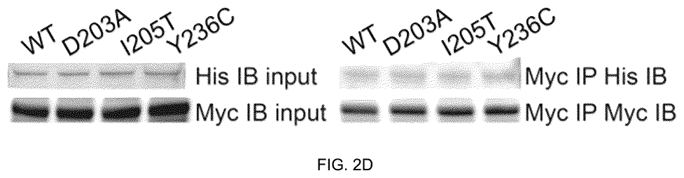

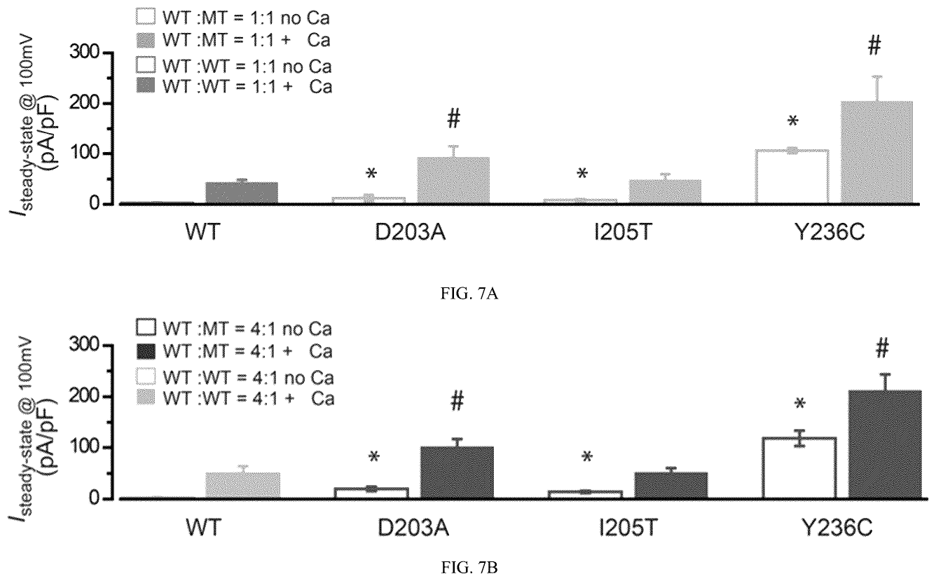

Three BEST1 gain-of-function mutations were previously identified, namely D203A, I205T and Y236C, all of which are autosomal dominant. To test whether their behavior is dominant in vitro, each mutant was individually co-expressed with WT at 1:1 in HEK293 cells and subjected to patch clamp analysis. Without Ca²⁺, Cl⁻ currents from cells co-expressing WT BEST1 and any of the mutants were significantly larger than those from cells expressing WT BEST1 only; at 1.2 μM [Ca²⁺]ᵢ, cells co-expressing D203A/WT and Y236C/WT displayed significantly bigger currents than WT only (FIGS. 2A-2C, left, and FIG. 7A); at both conditions, currents from cells co-expressing mutant/WT BEST1 resembled those from cells expressing the mutant only (FIGS. 2A-2C, left, and FIG. 7A). These results indicated that these three gain-of-function mutations were indeed dominant, in contrast to the dominant-negative behavior of the six loss-of-function mutations.

Since BEST1 is presumably a pentamer based on known bestrophin structures, it was possible that as few as one gain-of-function mutant monomer in the pentameric assembly could alter the channel function. HEK293 cells were co-transfected with mutant/WT BEST1 at a 1:4 ratio for patch clamp analysis. Under this condition, Ca²⁺-dependent Cl⁻ currents from co-expression of a gain-of-function mutant and WT BEST1 were still similar to those from the mutant only (FIGS. 2A-2C, right, and FIG. 7B). These results suggested a potent dominant effect of the gain-of-function mutations: just one mutant monomer was sufficient to dominate the function of the pentameric channel. To confirm the interaction between the gain-of-function mutant and WT monomers, mutant BEST1-YFP-His and WT BEST1-CFP-Myc were co-expressed in HEK293 cells, followed by immunoprecipitation with an antibody against Myc and immunoblotting with antibodies against His and Myc, respectively. All three gain-of-function mutants were expressed at similar levels to that of WT BEST1 after transient transfection, and retained the interaction with WT BEST1 (FIG. 2D), consistent with the previous observation that the interaction between BEST1 monomers was not affected by loss-of-function autosomal dominant mutations.

### EXAMPLE 4

### Modeling BEST1 Gain-of-Function Mutations in hPSC-RPE Cells

WT gene augmentation was sufficient to restore Ca²⁺-dependent Cl⁻ currents in iPSC-RPE cells with a BEST1 loss-of-function mutation, while the exogenous BEST1 was expressed at a comparable level to the endogenous protein. As BEST1 gain-of-function mutations are dominant over the WT even at a 1:4 ratio (FIGS. 2A-2C, right, and FIG. 7B), it raised an important question on the efficacy of gene augmentation. However, iPSC-RPE cells bearing a gain-of-function mutation are currently unavailable due to the lack of patient samples.

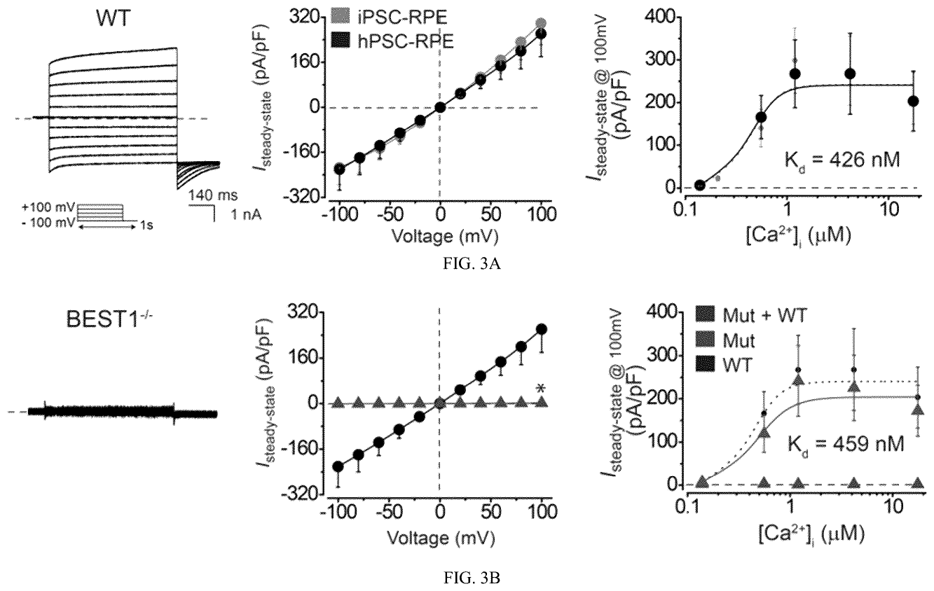

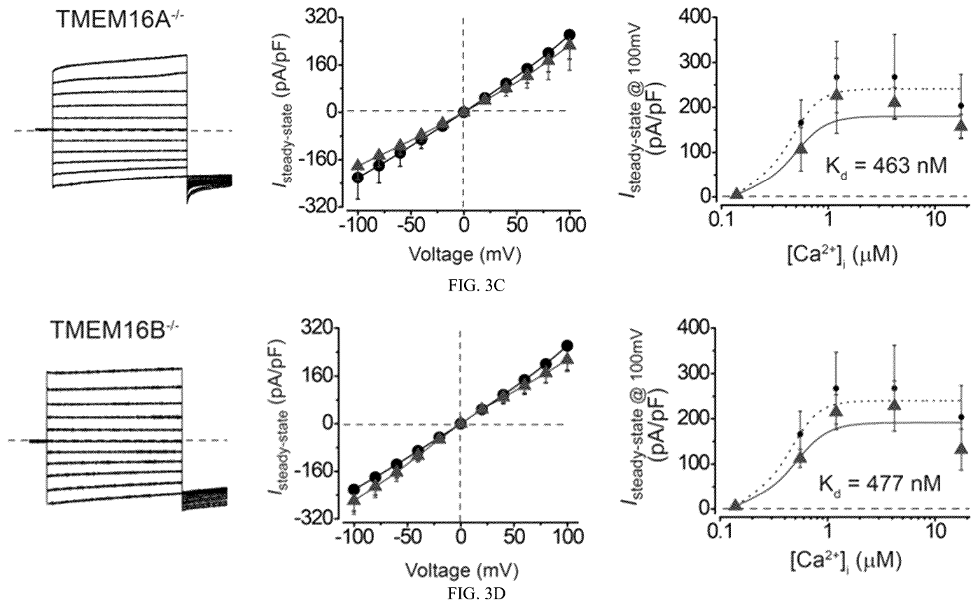

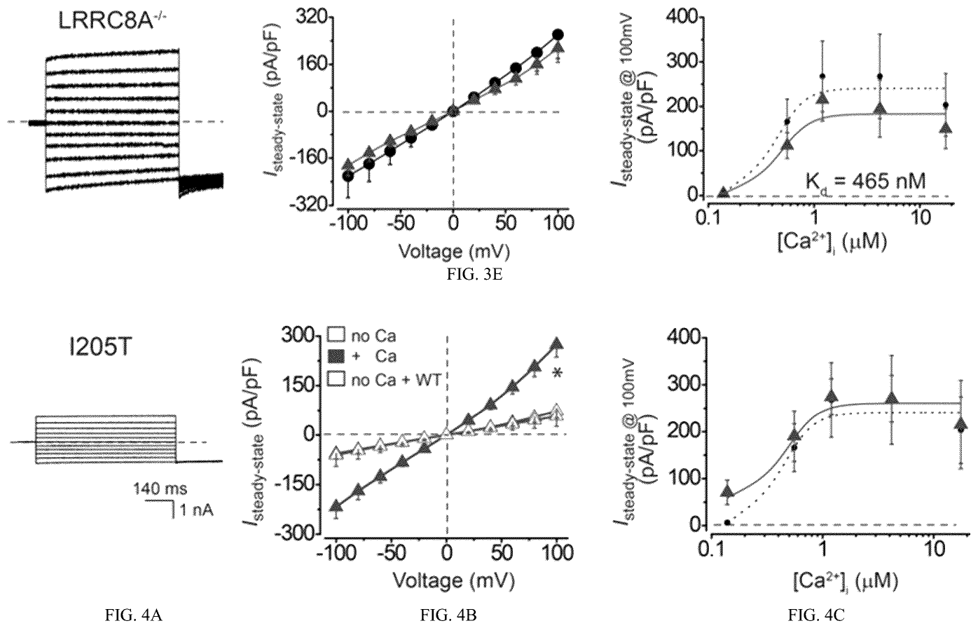

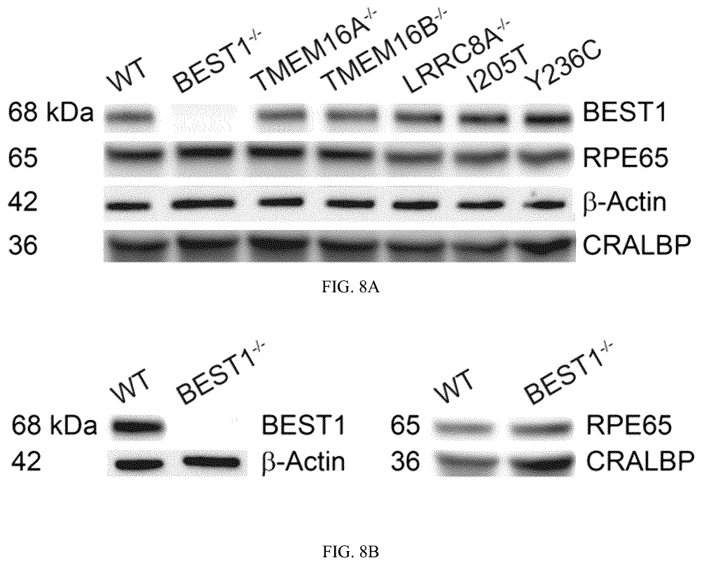

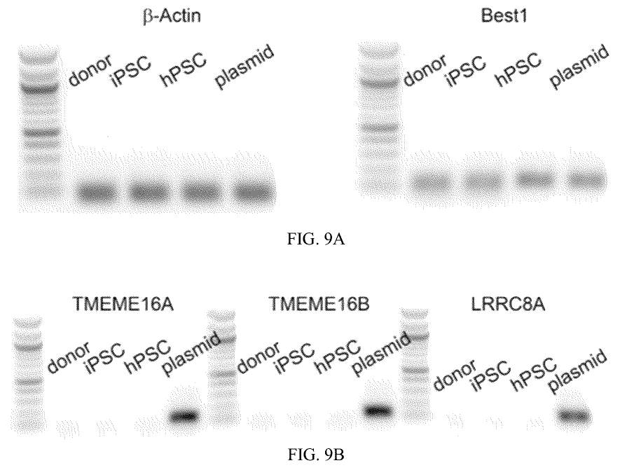

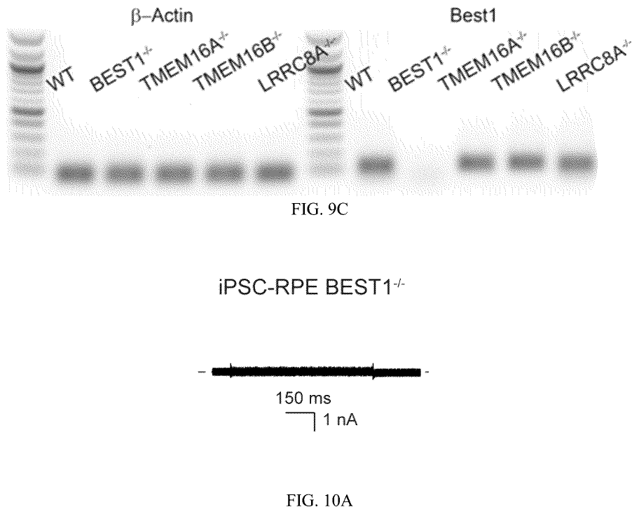

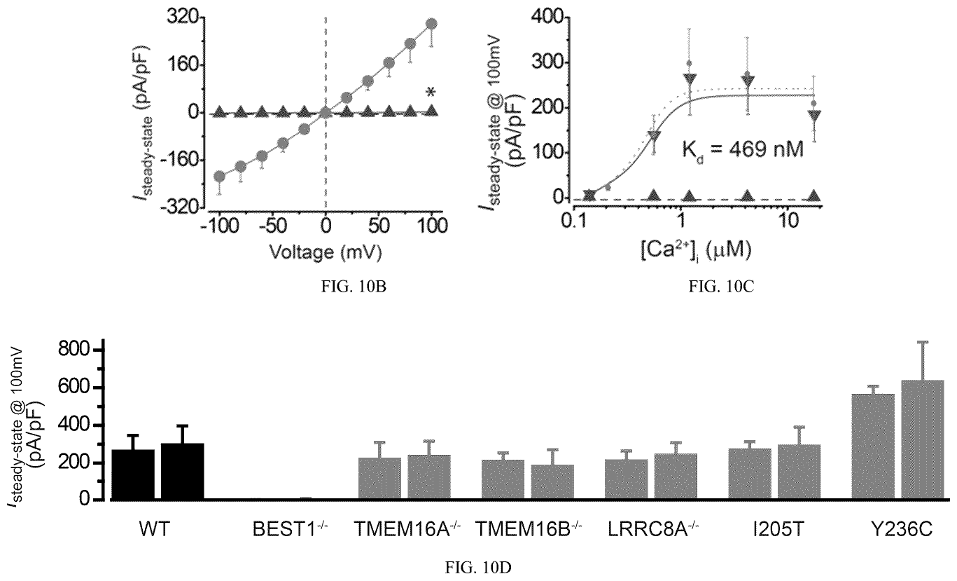

To circumvent this obstacle, isogenic RPE cells (hPSC-RPE) were generated from an H1 background hPSC line carrying an inducible Cas9 cassette (H1-iCas9), which allowed efficient genome editing. The RPE status of the hPSC-RPE cells was recognized by morphological signatures including intracellular pigment and hexagonal shape, and confirmed by immunoblotting with RPE-specific marker proteins RPE65 (retinal pigment epithelium-specific kDa protein) and CRALBP (cellular retinaldehyde-binding protein) (FIG. 8A), consistent with the result from donor-derived iPSC-RPE (FIG. 8B). Ca²⁺-dependent Cl⁻ currents on the plasma membrane of BEST1^{WT/WT} hPSC-RPE cells were recorded as 4±1 pA/pF and 267±79 pA/pF at 0 and 1.2 μ[Ca²⁺]ᵢ, respectively, consistent with results from donor-derived BEST1^{WT/WT} iPSC-RPE (FIG. 3A). To evaluate the genetic dependency of Ca²⁺-dependent Cl⁻ currents on BEST1 in RPE cells, BEST1 and three other CaCCs, namely TMEM16A, TMEM16B and LRRC8A in the H1-iCas9 cell line (Table 2), were individually knocked out and the corresponding knockout hPSC-RPE cells were generated for patch clamp analysis. It should be noted that only the mRNA of BEST1, but not of the other three CaCCs, can be detected in WT PSC-RPE or donor native RPE cells (FIGS. 9A-9B). Remarkably, Ca²⁺-dependent Cl⁻ current was completely eliminated in BEST1^{-/-} hPSC-RPE and a patient-derived BEST1 null (genotype) iPSC-RPE (FIG. 3B and FIGS. 10A-10D), in contrast to the WT-like currents from TMEM16A^{-/-}, TMEM16B^{-/-} or LRRC8A^{-/-} hPSC-RPE cells (FIGS. 3C-3E and 10D). Consistently, the protein and mRNA levels of BEST1 were abolished in BEST1^{-/-} hPSC-RPE cells, but not affected in TMEM16A^{-/-} TMEM16B^{-/-} or LRRC8A^{-/-} hPSC-RPE cells (FIGS. 8A and 9C). Moreover, when WT BEST1 was expressed from a baculovirus vector in BEST1^{-/-} hPSC-RPE and the patient-derived BEST1 null iPSC-RPE, Ca²⁺-dependent Cl⁻ currents were fully rescued (FIGS. 3B and 10C). Taken together, these results validated hPSC-RPE as a model system to study BEST1 function, and indicate that BEST1, but not TMEM16A, TMEM16B or LRRC8A, is the CaCC conducting Ca²⁺-dependent Cl⁻ current in human RPE.

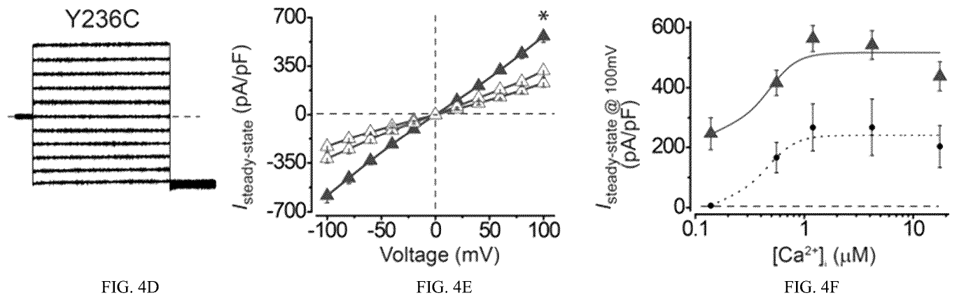

To model gain-of-function mutations, a heterozygous I205T and Y236C mutation was individually introduced to the BEST1 gene in the H1-iCas9 cell line, generating BEST^{I205T/WT} and BEST^{Y236C/WT} hPSC cells, which were then differentiated to BEST1^{I205T/WT} and BEST1^{Y236C/WT} hPSC-RPE cells, respectively, for patch clamp analysis (FIG. 8A). Consistent with results from transiently transfected HEK293 cells, Cl⁻ currents from BEST1^{I205T/WT} hPSC-RPE were significantly bigger than those from WT in the absence of Ca²⁺, but similar in the presence of Ca²⁺ (FIG. 4A-4C and 10D). On the other hand, the Ca²⁺-dependent Cl⁻ currents from BEST1^{Y236C/WT} hPSC-RPE were significantly larger than those from WT at all tested [Ca²⁺]ᵢs (FIGS. 4D-4F and 10D). These results reaffirmed the gain-of-function and dominant behavior of the BEST1 I205T and Y236C mutations in RPE.

### EXAMPLE 5

### BEST1 Gain-of-Function Mutations Cannot Be Rescued by Gene Augmentation in hPCS-RPE

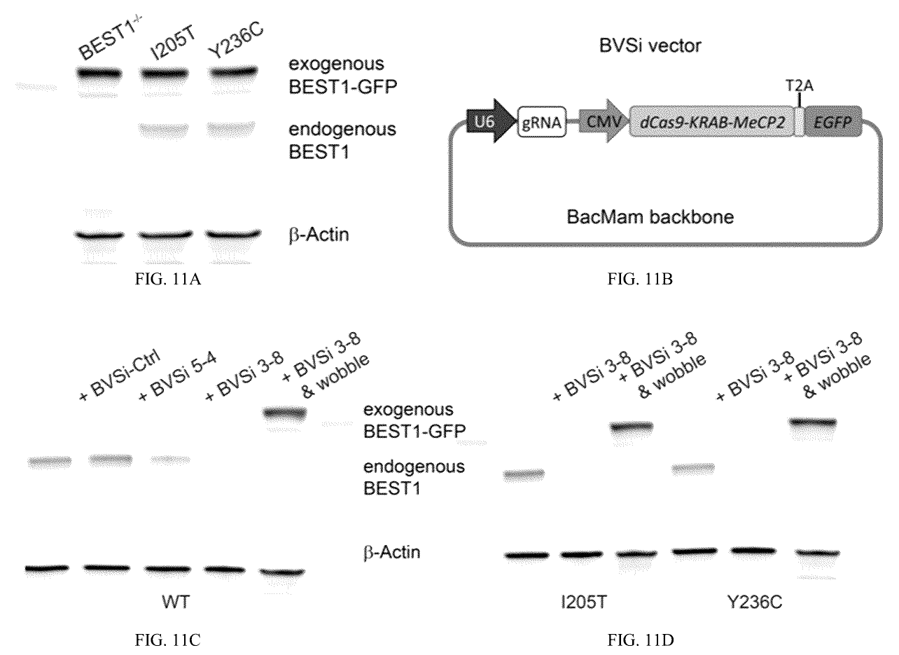

To test if the aberrant Ca²⁺-dependent Cl⁻ current in hPSC-RPE bearing a BEST1 gain-of-function mutation is rescuable by gene augmentation, BEST1^{I205T/WT} and BEST1^{Y236C/WT} hPSC-RPE cells were infected with baculoviruses expressing WT BEST1-GFP and subjected to patch clamp analysis. Notably, Ca²⁺-dependent Cl³¹ currents in these mutant hPSC-RPE cells remained aberrant after gene augmentation in the absence of Ca²⁺ (FIGS. 4B and 4E), despite that the exogenous WT BEST1 was expressed at a higher level to that of the endogenous BEST1 (FIG. 11A). This is in sharp contrast to the restoration of Ca²⁺-dependent Cl⁻ current in BEST1^{-/-} (FIGS. 3B and 11A) or loss-of-function mutant RPE cells using the same approach. Therefore, the results suggested that gene augmentation alone was insufficient to rescue BEST1 gain-of-function mutations.

### EXAMPLE 6

### Rescue of BEST1 Gain-of-Function Mutations by Unbiased CRISPR/Cas9-Mediated Gene Silencing in Combination with Augmentation

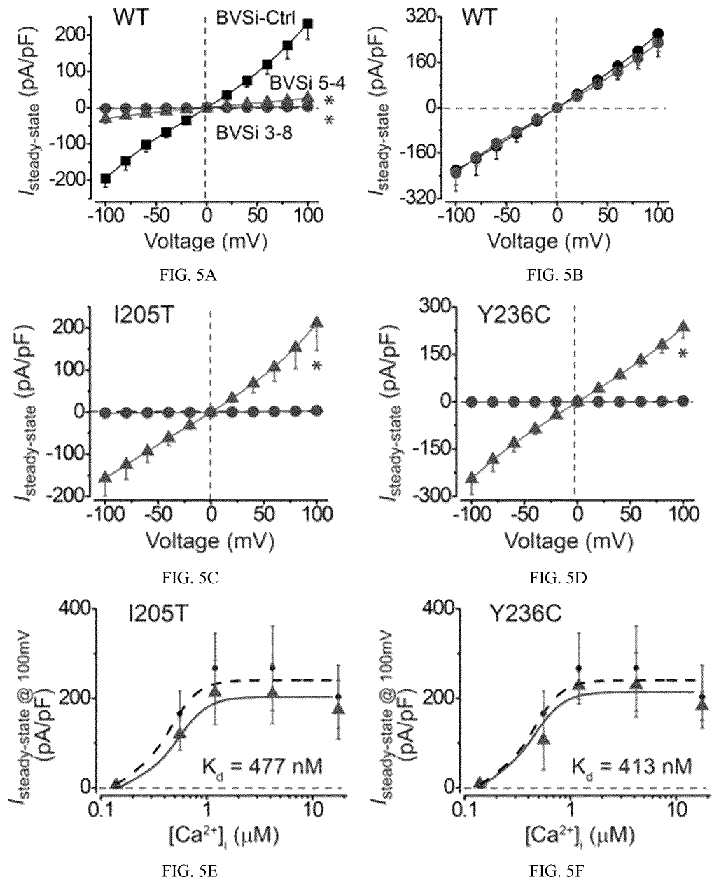

For the targeted silencing of endogenous BEST1, a programmable transcriptional repressor composed of a nuclease-dead Cas9 (dCas9) fused with a bipartite KRAB-MeCP2 repressor domain in the C-terminus (dCas9-KRAB-MeCP2) was employed. For the simultaneous delivery of the complete CRISPR machinery, a baculovirus-based silencing (BVSi) vector containing a CMV promoter driven dCas9-KRAB-MeCP2-T2A-GFP expression cassette and a U6 promoter driven gRNA expression cassette was constructed (FIG. 11B). Multiple guides targeting Exons 3 and 5 of BEST1 were screened by nuclease surveyor assay, and the most efficient ones along with a non-specific scramble guide were individually constructed into the BVSi backbone for virus production (Table 1). The resultant BEST1-targeting (BVSi 3-8 and BVSi 5-4) and control (BVSi-Ctrl) viruses were used to infect WT hPSC-RPE cells. Immunoblotting showed a better BEST1 knockdown efficiency of the BVSi 3-8 virus compared the BVSi 5-4 virus (FIG. 11C). Consistently, Ca²⁺-dependent Cl⁻ current from BVSi 3-8 infected cells was more effectively diminished compared to that from BVSi 5-4 infected cells at 1.2 μM [Ca²⁺]ᵢ (FIG. 5A), where RPE cells display the peak Cl⁻ current amplitude. These results indicated a high silencing efficacy of the BVSi 3-8 design, which was used for later steps of the silencing/augmentation strategy.

For augmentation of WT BEST1 in the presence of BVSi 3-8, a baculovirus bearing a wobble WT BEST1-mCherry resistant to the recognition by gRNA 3-8 was generated (FIG. 11C). When wobble WT BEST1-mCherry was co-expressed, the diminished Ca²⁺-dependent Cl⁻ current in BVSi 3-8 treated WT hPSC-RPE cells was fully rescued at 1.2 μM [Ca²⁺]ᵢ (FIG. 5B), validating the silencing/augmentation system in WT hPSC-RPE cells. To test this strategy for the rescue of gain-of-function mutations, the same set of experiments was carried out in BEST1^{I205T/WT} and BEST1^{Y236C/WT} hPSC-RPE cells. Remarkably, the endogenous BEST1 protein was diminished with BVSi 3-8 treatment (FIG. 11D) in the mutant hPSC-RPE cells, concomitant with abolished Ca²⁺-dependent Cl⁻ currents in these cells at 1.2 μM [Ca²⁺]ᵢ (FIGS. 5C-5D), while co-expression of the wobble WT BEST1-mCherry restored Cl⁻ currents to the WT levels at all tested [Ca²⁺]ᵢs (FIGS. 5C-5F and 11D), providing a proof-of-concept for the cure of Best disease associated with BEST1 gain-of-function mutations.

In this study, the influence of 10 patient-derived BEST1 mutations was compared, including one autosomal recessive mutation, six autosomal dominant loss-of-function mutations and three autosomal dominant gain-of-function mutations, on the channel activity of BEST1 in transiently transfected HEK293 cells. Although the recessive mutations and gain-of-function mutations exhibited their expected recessive and dominant behaviors, respectively, the autosomal dominant loss-of-function mutations only dominated over the WT BEST1 at a superior 4:1 ratio, but not at a canonical 1:1 ratio (FIG. 1). As the majority of the over 250 documented BEST1 disease-causing mutations are autosomal dominant and display loss-of-function when tested in vitro, the results indicated an important role of allele-specific epigenetic control in the development of bestrophinopathies. In strong support of this idea, imbalanced transcription of the two endogenous BEST1 alleles were detected in donor-derived iPSC-RPE and native RPE cells (Table 2), consistent with the previous observation that BEST1 was one of the inherited retinal disease genes with allelic expression imbalance (AEI) in the human retinal transcriptome.

AEI has been proven to be a common phenomenon in mammals. An SNP-array-based investigation surveyed 602 human genes and discovered more than half of genes displaying AEI, while a study by analyzing mouse transcriptome revealed that ˜20% of genes are prone to AEI in a tissue-specific manner. Moreover, AEI of somatic mutations has been well documented in the context of cancer, representing an important mechanism of tumorigenesis. However, the implication of AEI in inherited monogenic diseases is still poorly understood. There is no previous report of any inherited mutation which requires AEI for pathogenesis. Here, it was shown that Best disease due to dominant mutation of BEST1 in human RPE cells may serve as a paradigm to address the influence of AEI in Mendelian disorders.

Conventionally, autosomal dominant mutations are identified when the mutation is present on just one of the two BEST1 alleles in a bestrophinopathy patient. However, this definition only takes into account the genomic gene dosage, but not the allelic transcription/expression level. For instance, the six autosomal dominant loss-of-function mutations tested in this study all behaved recessively in HEK293 cells when co-transfected with the WT BEST1 at a 1:1 ratio, whereas the significantly decreased BEST1 channel activity in patient-derived iPSC-RPE cells was associated with a higher transcription level of the mutant allele compared to the WT allele, reflecting a dominant-negative effect rather than a canonical dominant effect. Therefore, at least a portion of the previously defined autosomal dominant mutations are de facto recessive, but exhibit a dominant-negative phenotype when their expression outweighs that of the WT BEST1 allele. This was in accord with the previous finding that gene augmentation was sufficient to rescue BEST1 loss-of-function mutations regardless of whether they are genetically classified as autosomal dominant or recessive, and provided an explanation for incomplete penetrance and variable clinical expressivity in patients bearing the same BEST1 mutation.

BEST1's intrinsic functionality as a CaCC, physiological localization in RPE and pathological relevance to retinal degenerative bestrophinopathies strongly suggested that BEST1 was the primary CaCC in RPE. Consistent with this idea, an indispensable role of BEST1 in generating Ca²⁺-dependent Cl⁻ currents in donor-derived iPSC-RPE cells was previously reported. However, other candidates, including TMEM16A and TMEM16B, have also been proposed to be the physiological CaCC(s) in porcine or primary mouse RPE and the human RPE-derived ARPE-19 cells. The results from isogenic knockout hPSC-RPE cells showed that Ca²⁺-dependent Cl⁻ currents were diminished in BEST1^{-/-} cells, and remained intact in TMEM16A^{-/-}, TMEM16B^{-/-} or LRRC8A^{-/-} cells (FIG. 3). Therefore, it was concluded that BEST1 is the bona fide CaCC in human RPE.

A “disease-in-a-dish” model, in which iPSC lines are reprogrammed from skin cells of different BEST1 mutation carriers was previously established and then differentiated into the corresponding iPSC-RPE cells for functional studies (FIG. 3A). This iPSC-RPE based model retained the patients' genetic background and thus had direct relevance to BEST1-associated retinal diseases, but was limited by the availability of patient samples. For instance, some BEST1 mutations are rarer than others, and the carrier(s) may not be willing or logistically feasible to provide a sample. Here, the “disease-in-a-dish” model has been further extended based on an engineered hPSC line (H1-iCas9), which allowed introduction of desired BEST1 mutations via CRISPR/Cas9-mediated genome editing technique, generating isogenic hPSC lines which can be differentiated into isogenic hPSC-RPE cells (FIGS. 3-4). Importantly, BEST1^{WT/WT} hPSC-RPE conducted almost identical Ca²⁺-dependent Cl⁻ currents as those from BEST1^{WT/WT} iPSC-RPE (FIG. 3A), validating hPSC-RPE as a versatile tool to model BEST1 mutations.

As the BEST1 channel is a pentameric assembly, the number of mutant protomer required for displaying a phenotype could theoretically be 1, 2, 3, 4 or 5. Interestingly, five subtypes of bestrophinopathies have been documented, underscoring a potential correlation between the “strength” of the mutations and the resultant disease. Supporting this hypothesis, ARB is specifically caused by BEST1 autosomal recessive mutations, which represent the “weakest” class that required five mutant protomers in a channel pentamer to be phenotypic (FIGS. 1H and 6). On the other hand, gain-of-function mutations such as D203A, I205T and Y236C represented the “strongest” class, which dominants over the WT BEST1 even at a 1:4 ratio (presumably one protomer per channel, FIGS. 2A-2C and 7B), although it remains unclear if they are specifically linked to a certain type of bestrophinopathy. Autosomal dominant loss-of-function mutations likely represent the “middle” classes, which require 2-4 protomers in a BEST1 channel to display the mutant phenotype. For instance, the six loss-of-function mutations tested in this study (A10T, R218H, L234P, A243T, Q293K and D302A) may represent the 4-mutant-protomer class as they are only dominant-negative at a 4:1 ratio to the WT in HEK293 cells, while Y85H, R92C, R218S and G299E may represent the ⅔-mutant-protomer class(es), as they were previously shown to dominant over the WT at a 1:1 ratio in HEK293 cells. However, it should be noted that the endogenous BEST1 mutant to WT molecule ratio in the RPE of bestrophinopathy patients with autosomal dominant mutations is still unknown because most of BEST1 mutations are missense, such that the WT and mutant proteins cannot be distinguished by antibody in immunoblotting.

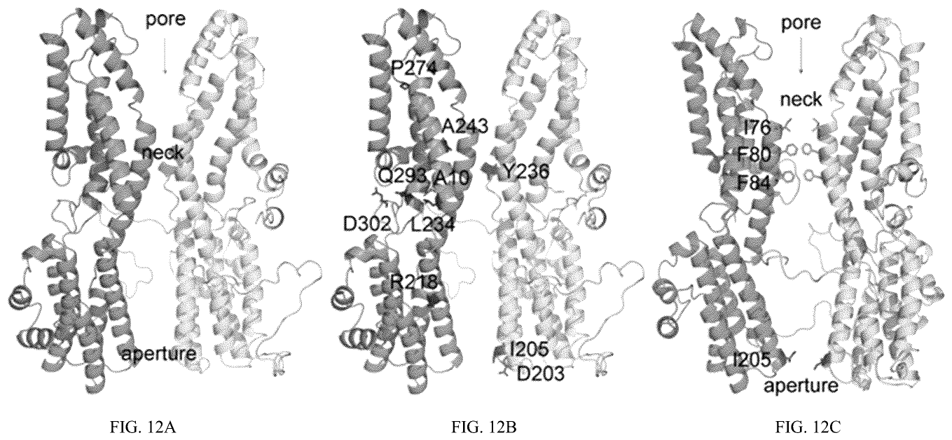

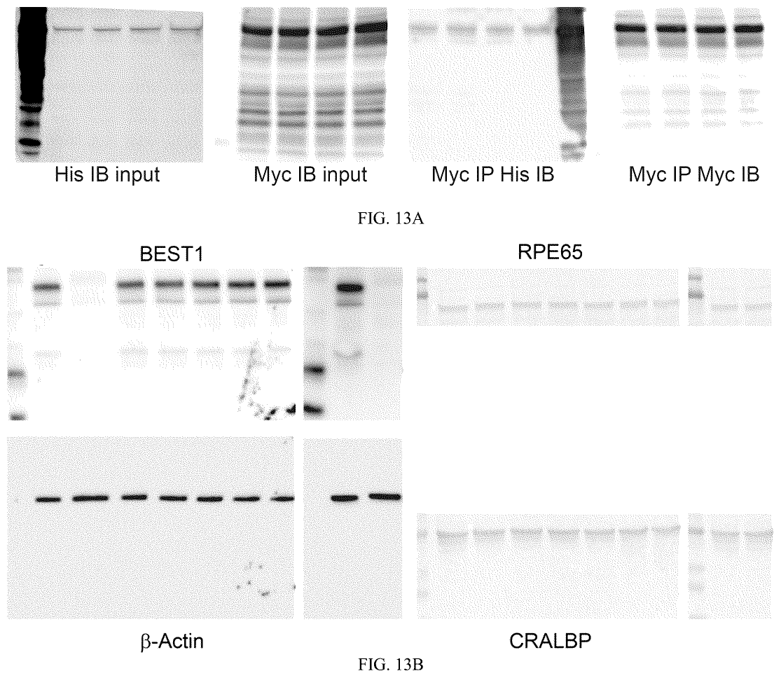

Gain-of-function mutations often have a strong dominant effect, consistent with the results that they are suppressing the WT even at a 1:4 ratio (FIGS. 2A-2C and 7B). This suggested that for effective gene augmentation therapy, the combined protein level of endogenous and exogenously supplied functional BEST1 must be at least four folds higher than that of the endogenous mutant BEST1. However, it was shown that even with a strong CMV promoter, which produced an apparently higher level of exogenous BEST1 protein compared to that of endogenous BEST1, the gain-of-function phenotype in BEST1^{I205T/WT} and BEST1^{Y236C/WT} hPSC-RPE cells cannot be rescued (FIGS. 4B, 4E and 11A). Therefore, it may be impractical to rescue BEST1 gain-of-function mutations by gene augmentation alone, as clinical applications would presumably require the native BEST1 promotor, which is even weaker than the CMV promotor. Structurally, the three gain-of-function mutations (D203A, 1205T and Y236C) are located at or in a dose proximity to the neck or the aperture of the channel (FIG. 12), and are involved in the opening of at least one of these two Ca²⁺-dependent gates. By contrast, loss-of-function mutations are located in various regions of the channel.

Unbiased suppression of both endogenous alleles in combination with WT gene augmentation was applied in this study using a CRISPR/Cas9-based gene silencing vector (BVSi) to suppress the endogenous BEST1 expression. As the BEST1 genomic locus recognized by the BVSi does not have any reported disease-causing mutations or polymorphisms, this BVSi design was universally suited for BEST1 silencing in bestrophinopathy patients no matter where their mutations are located. Notably, although gene augmentation alone was readily sufficient to rescue loss-of-function mutations, simultaneously suppressing the endogenous BEST1 does not interfere with the functional restoration. Therefore, the silencing+augmentation combo strategy is contemplated as an optimal approach for the treatment of bestrophinopathies.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

### REFERENCES

1 Marquardt, A. et al. Mutations in a novel gene, VMD2, encoding a protein of unknown properties cause juvenile-onset vitelliform macular dystrophy (Best's disease). Human molecular genetics 7, 1517-1525, doi:ddb199 [pii] (1998).
2 Petrukhin, K. et al. Identification of the gene responsible for Best macular dystrophy. Nature genetics 19, 241-247, doi:10.1038/915 (1998).
3 Burgess, R. et al. Biallelic mutation of BEST1 causes a distinct retinopathy in humans. American journal of human genetics 82, 19-31, doi:10.1016/j.ajhg.2007.08.004 (2008).
4 Allikmets, R. et al. Evaluation of the Best disease gene in patients with age-related macular degeneration and other maculopathies. Hum Genet 104, 449-453 (1999).
5 Kramer, F. et al. Mutations in the VMD2 gene are associated with juvenile-onset vitelliform macular dystrophy (Best disease) and adult vitelliform macular dystrophy but not age-related macular degeneration. Eur J Hum Genet 8, 286-292, doi:10.1038/sj.ejhg.5200447 (2000).
6 Yardley, J. et al. Mutations of VMD2 splicing regulators cause nanophthalmos and autosomal dominant vitreoretinochoroidopathy (ADVIRC). Investigative ophthalmology & visual science 45, 3683-3689, doi:10.1167/iovs.04-0550 (2004).
7 Davidson, A. E. et al. Missense mutations in a retinal pigment epithelium protein, bestrophin-1, cause retinitis pigmentosa. American journal of human genetics 85, 581-592, doi:10.1016/j.ajhg.2009.09.015 (2009).
8 Johnson, A. A. et al. Bestrophin 1 and retinal disease. Progress in retinal and eye research, doi:10.1016/j.preteyeres.2017.01.006 (2017).
9 Yang, T., Justus, S., Li, Y. & Tsang, S. H. BEST1: the Best Target for Gene and Cell Therapies. Molecular therapy: the journal of the American Society of Gene Therapy 23, 1805-1809, doi:10.1038/mt.2015.177 (2015).
10 Marmorstein, A. D. et al. Bestrophin, the product of the Best vitelliform macular dystrophy gene (VMD2), localizes to the basolateral plasma membrane of the retinal pigment epithelium. Proc Natl Acad Sci USA 97, 12758-12763, doi:10.1073/pnas.220402097 [pii] (2000).
11 Li, Y. et al. Patient-specific mutations impair BESTROPHIN1's essential role in mediating Ca2+-dependent Cl− currents in human RPE. Elife 6, doi:10.7554/eLife.29914 (2017).
12 Yang, T. et al. Structure and selectivity in bestrophin ion channels. Science 346, 355-359, doi:10.1126/science.1259723 (2014).
13 Kane Dickson, V., Pedi, L. & Long, S. B. Structure and insights into the function of a Ca(2+)-activated Cl(−) channel. Nature 516, 213-218, doi:10.1038/nature13913 (2014).
14 Owji, A. P. et al. Structural and functional characterization of the bestrophin-2 anion channel. Nat Struct Mol Biol 27, 382-391, doi:10.1038/s41594-020-0402-z (2020).
15 Hartzell, H. C., Qu, Z., Yu, K., Xiao, Q. & Chien, L. T. Molecular physiology of bestrophins: multifunctional membrane proteins linked to best disease and other retinopathies. Physiol Rev 88, 639-672, doi:10.1152/physrev.00022.200788/2/639 [pii] (2008).
16 Johnson, A. A. et al. Disease-causing mutations associated with four bestrophinopathies exhibit disparate effects on the localization, but not the oligomerization, of Bestrophin-1. Experimental eye research 121, 74-85, doi:10.1016/j.exer.2014.02.006 (2014).
17 Ji, C. et al. Dual Ca(2+)-dependent gates in human Bestrophinl underlie disease-causing mechanisms of gain-of-function mutations. Commun Biol 2, 240, doi:10.1038/s42003-019-0433-3433 [pii] (2019).
18 Ji, C. et al. Investigation and Restoration of BEST1 Activity in Patient-derived RPEs with Dominant Mutations. Scientific reports 9, 19026, doi:10.1038/s41598-019-54892-7 (2019).
19 Llavona, P. et al. Allelic Expression Imbalance in the Human Retinal Transcriptome and Potential Impact on Inherited Retinal Diseases. Genes 8, doi:10.3390/genes8100283 (2017).
20 Moshfegh, Y. et al. BESTROPHIN1 mutations cause defective chloride conductance in patient stem cell-derived RPE. Human molecular genetics 25, 2672-2680, doi:10.1093/hmg/ddw126 (2016).
21 Idelson, M. et al. Directed differentiation of human embryonic stem cells into functional retinal pigment epithelium cells. Cell stem cell 5, 396-408, doi:10.1016/j.stem.2009.07.002 (2009).
22 Yeo, N. C. et al. An enhanced CRISPR repressor for targeted mammalian gene regulation. Nature methods 15, 611-616, doi:10.1038/s41592-018-0048-5 (2018).
23 Yan, H., Yuan, W., Velculescu, V. E., Vogelstein, B. & Kinzler, K. W. Allelic variation in human gene expression. Science 297, 1143, doi:10.1126/science.1072545 (2002).
24 Lo, H. S. et al. Allelic variation in gene expression is common in the human genome. Genome research 13, 1855-1862, doi:10.1101/gr.1006603 (2003).
25 Pinter, S. F. et al. Allelic Imbalance Is a Prevalent and Tissue-Specific Feature of the Mouse Transcriptome. Genetics 200, 537-549, doi:10.1534/genetics.115.176263 (2015).
26 Bielski, C. M. et al. Widespread Selection for Oncogenic Mutant Allele Imbalance in Cancer. Cancer cell 34, 852-862 e854, doi:10.1016/j.cce11.2018.10.003 (2018).
27 Rhee, J. K., Lee, S., Park, W. Y., Kim, Y. H. & Kim, T. M. Allelic imbalance of somatic mutations in cancer genomes and transcriptomes. Scientific reports 7, 1653, doi:10.1038/s41598-017-01966-z (2017).
28 Yan, H. et al. Small changes in expression affect predisposition to tumorigenesis. Nature genetics 30, 25-26, doi:10.1038/ng799 (2002).
29 C.M., B. & B.S., T. Mutant Allele Imbalance in Cancer. Annu. Rev. Cancer Biol. (2021).
30 Sodi, A. et al. BEST1 sequence variants in Italian patients with vitelliform macular dystrophy. Molecular vision 18, 2736-2748 (2012).
31 Cohn, A. C. et al. Best's macular dystrophy in Australia: phenotypic profile and identification of novel BEST1 mutations. Eye 25, 208-217, doi:10.1038/eye.2010.180 (2011).
32 Arora, R. et al. Unilateral BEST1-Associated Retinopathy. Am J Ophthalmol 169, 24-32, doi:10.1016/j.ajo.2016.05.024 (2016).
33 Schreiber, R. & Kunzelmann, K. Expression of anoctamins in retinal pigment epithelium (RPE). Pflugers Archiv: European journal of physiology 468, 1921-1929, doi:10.1007/s00424-016-1898-2 (2016).
34 Keckeis, S., Reichhart, N., Roubeix, C. & Strauss, O. Anoctamin2 (TMEM16B) forms the Ca2+-activated Cl− channel in the retinal pigment epithelium. Experimental eye research 154, 139-150, doi:10.1016/j.exer.2016.12.003 (2016).
35 Kittredge, A., Ji, C., Zhang, Y. & Yang, T. Differentiation, Maintenance, and Analysis of Human Retinal Pigment Epithelium Cells: A Disease-in-a-dish Model for BEST1 Mutations. Journal of visualized experiments: JoVE, doi:10.3791/57791 (2018).
36 Sun, H., Tsunenari, T., Yau, K. W. & Nathans, J. The vitelliform macular dystrophy protein defines a new family of chloride channels. Proc Natl Acad Sci USA 99, 4008-4013, doi:10.1073/pnas.05269299999/6/4008 [pii] (2002).
37 Zhang, Y. et al. ATP activates bestrophin ion channels through direct interaction. Nature communications 9, 3126, doi:10.1038/s41467-018-05616-410.1038/s41467-018-05616-4 [pii] (2018).
38 Li, Y., Nguyen, H. V. & Tsang, S. H. Skin Biopsy and Patient-Specific Stem Cell Lines. Methods Mot Blot 1353, 77-88, doi:10.1007/7651 2015 225 (2016).
39 Maminishkis, A. et al. Confluent monolayers of cultured human fetal retinal pigment epithelium exhibit morphology and physiology of native tissue. Investigative ophthalmology & visual science 47, 3612-3624, doi:10.1167/iovs.05-1622 (2006).
40 Yang, T., Hendrickson, W. A. & Colecraft, H. M. Preassociated apocalmodulin mediates Ca2+-dependent sensitization of activation and inactivation of TMEM16A/16B Ca2+-gated Cl− channels. Proc Natl Acad Sci USA 111, 18213-18218, doi:10.1073/pnas.1420984111 (2014).
41 Goehring, A. et al. Screening and large-scale expression of membrane proteins in mammalian cells for structural studies. Nature protocols 9, 2574-2585, doi:10.1038/nprot.2014.173 (2014).
42 Zhu, Z., Gonzalez, F. & Huangfu, D. The iCRISPR platform for rapid genome editing in human pluripotent stem cells. Methods in enzymology 546, 215-250, doi:10.1016/B978-0-12-801185-0.00011-8 (2014).
43 Yang, T., He, L. L., Chen, M., Fang, K. & Colecraft, H. M. Bio-inspired voltage-dependent calcium channel blockers. Nature communications 4, 2540, doi:10.1038/ncomms3540 (2013).
44 Yang, T., Suhail, Y., Dalton, S., Kernan, T. & Colecraft, H. M. Genetically encoded molecules for inducibly inactivating CaV channels. Nat Chem Biol 3, 795-804, doi: 10.1038/nchembio.2007.42 (2007).

1. A method comprising
introducing into a cell an effective amount of:
a Clustered Regularly Interspaced Short Palindromic Repeats (CRISPR) interference (CRISPRi) system, or one or more nucleic acids encoding the CRISPRi system, configured to knockout or at least partially silence both alleles of a target endogenous gene, wherein the CRISPRi system comprises: (a) at least one Cas protein, (b) at least one gRNA, wherein each gRNA is configured to hybridize to a portion of the nucleic acid sequence encoding the target endogenous gene and (c) a transcriptional repressor; and
a nucleic acid encoding an exogenous functional version of the target endogenous gene.

2. The method of claim 1, wherein the Cas protein and the transcriptional repressor are provided as a fusion protein, or a nucleic acid encoding thereof.

3. The method of claim 1 or claim 2, wherein the Cas protein is catalytically dead.

4. The method of any of claims 1-3, wherein the Cas protein is Cas9, Cas12a, and Cas14.

5. The method of any of claims 1-4, wherein the Cas protein, the at least one gRNA, and the transcriptional repressor are provided on a single nucleic acid.

6. The method of claim 5, wherein the single nucleic acid is a baculovirus or lentivirus vector.

7. The method of any of claims 1-6, wherein the target endogenous gene is a disease-associated gene.

8. The method of any of claims 1-7, wherein the cell is in vivo.

9. The method of claim 8, wherein the introducing into a cell comprises administering to a subject.

10. The method of claim 9, wherein the subject has or is suspected to have a disease or disorder selected from the group consisting of a neurodegenerative disease and an ocular disease.

11. The method of claim 10, wherein the disease or disorder comprises Best vitelliform macular dystrophy (BVMD), autosomal recessive bestrophinopathy (ARB), adult-onset vitelliform dystrophy (AVMD), autosomal dominant vitreoretinochoroidopathy (ADVIRC), or retinitis pigmentosa (RP).

12. The method of any of claims 1-11, wherein the target endogenous gene is BEST1.

13. The method of claim 12, wherein the BEST1 comprises a D203A, I205T or Y236C mutation.

14. A system comprising:
a Clustered Regularly Interspaced Short Palindromic Repeats (CRISPR)-Cas system, or one or more nucleic acids encoding the CRISPR-Cas system, configured to knockout or at least partially silence both alleles of a target endogenous gene, wherein the CRISPRi system comprises: (a) at least one Cas protein, (b) at least one gRNA, wherein each gRNA is configured to hybridize to a portion of the nucleic acid sequence encoding the target endogenous gene and (c) a transcriptional repressor; and
a nucleic acid encoding an exogenous functional version of the target endogenous gene.

15. The system of claim 14, wherein the target endogenous gene is BEST1.
